(19) United States
(12) Patent Application Publication
(10) Pub. No.: US 2022/0112258 A1
(43) Pub. Date: Apr. 14, 2022

(54) BIFUNCTIONAL MOLECULES WITH IL-7 ACTIVITY

(71) Applicant: I-Mab Biopharma US Limited, Gaithersburg, MD (US)

(72) Inventors: Feifei Cui, Shanghai (CN); Lei Fang, Shanghai (CN); Yuanyuan Yang, Shanghai (CN); Zhengyi Wang, Shanghai (CN); Bingshi Guo, Shanghai (CN)

(21) Appl. No.: 17/278,157

(22) PCT Filed: Jul. 27, 2020
(86) PCT No.: PCT/CN2020/104857

§ 371 Date: Mar. 19, 2021

(30) Foreign Application Priority Data
Jul. 25, 2019 (CN) PCT/CN2019/097772

(51) Int. Cl. C07K 14/54; C07K 16/28; A61P 35/00

(52) U.S. Cl. CPC C07K 14/5418; C07K 16/2827; A61P 35/00; C07K 2317/76; A61K 2039/505; C07K 2317/94; C07K 2319/33; C07K 2319/30; C07K 2317/92

(57) ABSTRACT

Provided are polypeptides having an antigen-binding unit targeting a tumor antigen and an IL-7 protein or fragment thereof, fused to a Fc fragment. The disclosed polypeptides can be used for treating cancer.

The present invention claims the priority of the PCT/CN2019/097772, filed on Jul. 25, 2019, the contents of which are incorporated herein by its entirety.

### BACKGROUND

Interleukin 7 (IL-7) is a hematopoietic growth factor secreted by stromal cells in the bone marrow and thymus. IL-7 stimulates the differentiation of multipotent hematopoietic stem cells into lymphoid progenitor cells. It also stimulates proliferation of all cells in the lymphoid lineage (B cells, T cells and NK cells). IL-7 is a cytokine important for B and T cell development. This cytokine and the hepatocyte growth factor (HGF) form a heterodimer that functions as a pre-pro-B cell growth-stimulating factor. This cytokine is found to be a cofactor for V(D)J rearrangement of the T cell receptor beta (TCRß) during early T cell development. This cytokine can be produced locally by intestinal epithelial and epithelial goblet cells, and may serve as a regulatory factor for intestinal mucosal lymphocytes.

Recombinant IL-7 has been safely administered to patients in several phase I and II clinical trials. A human study of IL-7 in patients with cancer demonstrated that administration of this cytokine can transiently disrupt the homeostasis of both CD8⁺ and CD4⁺ T cells with a commensurate decrease in the percentage of CD4⁺CD25⁺Foxp3⁺ T regulatory cells. No objective cancer regression, however, was observed.

### SUMMARY

The present disclosure compared different formats of bifunctional molecules which included one or more IL-7 protein or its variants and an antibody or antigen-binding fragment having specificity to a tumor antigen or immune checkpoint molecule (e.g., PD-L1). It is discovered that when the IL-7 protein or its variant is fused to the N-terminus of a Fc fragment while the antigen-binding fragment is fused to the N-terminus of another chain of the Fc fragment, the bifunctional molecule has the highest stability and excellent activities.

In accordance with one embodiment of the present disclosure, therefore, provided is a polypeptide having an antigen-binding unit having specificity to a tumor antigen or immune checkpoint molecule, and an IL-7 protein or homologue, both of which are fused to, preferably the N-terminus, of a Fc fragment. In some embodiments, provided is a polypeptide comprising: a first portion comprising, in an N-terminal to C-terminal order, a first fragment, a CH2 fragment, and a CH3 fragment, wherein the first fragment comprises an IL-7 protein, an IL-7 protein homologue having at least 75% sequence identity to the IL-7 protein, or a fragment thereof, and wherein the first fragment is capable of binding an IL-7 receptor; and a second portion comprising, in an N-terminal to C-terminal order, an antigen-binding fragment, a CH2 fragment, and a CH3 fragment, wherein the antigen-binding fragment is capable of specifically binding to tumor antigen or an immune checkpoint molecule, wherein the first portion is paired to the second portion through interaction between the CH2 fragments and/or between the CH3 fragments.

Methods of treating a cancer are also provided, in a patient in need thereof. In some embodiments, the method entails administering to the patient a molecule of the present disclosure. In some embodiments, the cancer is selected from the group consisting of bladder cancer, liver cancer, colon cancer, rectal cancer, endometrial cancer, leukemia, lymphoma, pancreatic cancer, small cell lung cancer, non-small cell lung cancer, breast cancer, urethral cancer, head and neck cancer, gastrointestinal cancer, stomach cancer, oesophageal cancer, ovarian cancer, renal cancer, melanoma, prostate cancer and thyroid cancer.

### BRIEF DESCRIPTION OF THE DRAWINGS

FIG. 1 shows concentration-time curves in serum and the pharmacokinetic parameters of tested bifunctional molecules.

FIG. 2 shows absolute cell counts following treatments by the bifunctional molecules.

FIG. 3, with panels A-B, shows changes to T cell proliferation measured by Ki67 expression on CD4⁺ or CD8⁺ T cells following treatments by the bifunctional molecules.

FIG. 4, with panels A-B, shows changes of percentages of P-STAT5⁺CD4⁺ T cells and IL7Rα expression levels following the indicated treatments.

FIG. 5 illustrates the structures of Formats 1, 2 and 3.

FIG. 6 shows the serum concentrations of intact molecule and the anti-PD-L1 part of L1I7 Formats 1, 2 and 3 after a single dose of the tested bifunctional molecule in mice.

FIG. 7 shows concentration-time curves in serum and the pharmacokinetic parameters of L1I7^{WT} after a single dose of the tested bifunctional molecule.

FIG. 8 shows that the absolute cell counts following treatment of L1I7^{WT} Format 2.

FIG. 9, with panels A-B, show that percentage changes of Ki67⁺CD4⁺ and Ki67⁺CD8⁺ T cells following L1I7^{WT} Format 2 treatment.

FIG. 10 show PD-L1 binding affinity of L1I7 Format 2 molecules measured by Biacore.

FIG. 11 show the ELISA (A-B) and cell binding (C-D) results for the bifunctional molecules analyzed for their binding to human PD-L1.

FIG. 12, with panels A-B, show the cell based functional assay validating the PDL1 antogonistic effect of the L1I7 fusion molecules.

FIG. 13, with panels A-B, shows changes of MFI of P-STAT5 in human CD4⁺ T cells following the indicated treatments.

FIG. 14, with panels A-D, shows IL-7R binding and ligation-induced internalization on human CD4⁺ T cells following the indicated treatments.

FIG. 15, with panels A-B, shows CD4⁺ T cell proliferation following the indicated treatments.

FIG. 16 shows in vitro T cell activation effect following the indicated treatments.

FIG. 17, with panels A-B, shows the in vivo tracking and tissue distribution of indicated molecules in PBMC-reconstituted HCC1954 tumor cell transplanted mice.

FIG. 18 shows the bispecific binding curve of L1I7 molecules to IL-7Rα and PD-L1.

FIG. 19 shows PK profile of L1I7 Format 2 series of molecules for the first dose in cynomolgus monkeys.

FIG. 20, with panels A-D, shows hematological parameters in cynomolgus monkeys following three consecutive intravenous injections of L1I7 series of molecules.

FIG. 21, with panels A-D, show T cell proliferation following three consecutive intravenous injections of L1I7 molecules in cynomolgus monkeys.

### DETAILED DESCRIPTION

### Definitions

It is to be noted that the term “a” or “an” entity refers to one or more of that entity; for example, “an antibody,” is understood to represent one or more antibodies. As such, the terms “a” (or “an”), “one or more,” and “at least one” can be used interchangeably herein.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

The term “isolated” as used herein with respect to cells, nucleic acids, such as DNA or RNA, refers to molecules separated from other DNAs or RNAs, respectively, that are present in the natural source of the macromolecule. The term “isolated” as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to cells or polypeptides which are isolated from other cellular proteins or tissues. Isolated polypeptides is meant to encompass both purified and recombinant polypeptides.

As used herein, the term “recombinant” as it pertains to polypeptides or polynucleotides intends a form of the polypeptide or polynucleotide that does not exist naturally, a non-limiting example of which can be created by combining polynucleotides or polypeptides that would not normally occur together.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, though preferably less than 25% identity, with one of the sequences of the present disclosure.

A polynucleotide or polynucleotide region (or a polypeptide or polypeptide region) has a certain percentage (for example, 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example those described in Ausubel et al. eds. (2007) Current Protocols in Molecular Biology.

Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Biologically equivalent polynucleotides are those having the above-noted specified percent homology and encoding a polypeptide having the same or similar biological activity.

The term “an equivalent nucleic acid or polynucleotide” refers to a nucleic acid having a nucleotide sequence having a certain degree of homology, or sequence identity, with the nucleotide sequence of the nucleic acid or complement thereof. A homolog of a double stranded nucleic acid is intended to include nucleic acids having a nucleotide sequence which has a certain degree of homology with or with the complement thereof. In one aspect, homologs of nucleic acids are capable of hybridizing to the nucleic acid or complement thereof. Likewise, “an equivalent polypeptide” refers to a polypeptide having a certain degree of homology, or sequence identity, with the amino acid sequence of a reference polypeptide. In some aspects, the sequence identity is at least about 70%, 75%, 80%, 85%, 90%, 95%, 98%, or 99%. In some aspects, the equivalent polypeptide or polynucleotide has one, two, three, four or five addition, deletion, substitution and their combinations thereof as compared to the reference polypeptide or polynucleotide. In some aspects, the equivalent sequence retains the activity (e.g., epitope-binding) or structure (e.g., salt-bridge) of the reference sequence.

Hybridization reactions can be performed under conditions of different “stringency”. In general, a low stringency hybridization reaction is carried out at about 40° C. in about 10×SSC or a solution of equivalent ionic strength/temperature. A moderate stringency hybridization is typically performed at about 50° C. in about 6×SSC, and a high stringency hybridization reaction is generally performed at about 60° C. in about 1×SSC. Hybridization reactions can also be performed under “physiological conditions” which is well known to one of skill in the art. A non-limiting example of a physiological condition is the temperature, ionic strength, pH and concentration of Mg²⁺ normally found in a cell.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching. The term “polymorphism” refers to the coexistence of more than one form of a gene or portion thereof. A portion of a gene of which there are at least two different forms, i.e., two different nucleotide sequences, is referred to as a “polymorphic region of a gene”. A polymorphic region can be a single nucleotide, the identity of which differs in different alleles.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, dsRNA, siRNA, miRNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this disclosure that is a polynucleotide encompasses both the double-stranded form and each of two complementary single-stranded forms known or predicted to make up the double-stranded form.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

As used herein, an “antibody” or “antigen-binding polypeptide” refers to a polypeptide or a polypeptide complex that specifically recognizes and binds to an antigen. An antibody can be a whole antibody and any antigen binding fragment or a single chain thereof. Thus the term “antibody” includes any protein or peptide containing molecule that comprises at least a portion of an immunoglobulin molecule having biological activity of binding to the antigen. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein.

The terms “antibody fragment” or “antigen-binding fragment”, as used herein, is a portion of an antibody such as F(ab′)₂, F(ab)₂, Fab′, Fab, Fv, scFv and the like. Regardless of structure, an antibody fragment binds with the same antigen that is recognized by the intact antibody. The term “antibody fragment” includes aptamers, spiegelmers, and diabodies. The term “antibody fragment” also includes any synthetic or genetically engineered protein that acts like an antibody by binding to a specific antigen to form a complex.

A “single-chain variable fragment” or “scFv” refers to a fusion protein of the variable regions of the heavy (V_{H}) and light chains (V_{L}) of immunoglobulins. In some aspects, the regions are connected with a short linker peptide of ten to about 25 amino acids. The linker can be rich in glycine for flexibility, as well as serine or threonine for solubility, and can either connect the N-terminus of the V_{H} with the C-terminus of the V_{L}, or vice versa. This protein retains the specificity of the original immunoglobulin, despite removal of the constant regions and the introduction of the linker. ScFv molecules are known in the art and are described, e.g., in U.S. Pat. No. 5,892,019.

The term antibody encompasses various broad classes of polypeptides that can be distinguished biochemically. Those skilled in the art will appreciate that heavy chains are classified as gamma, mu, alpha, delta, or epsilon (γ, μ, α, δ, ε) with some subclasses among them (e.g., γ1-γ4). It is the nature of this chain that determines the “class” of the antibody as IgG, IgM, IgA IgG, or IgE, respectively. The immunoglobulin subclasses (isotypes) e.g., IgG₁, IgG₂, IgG₃, IgG₄, IgG₅, etc. are well characterized and are known to confer functional specialization. Modified versions of each of these classes and isotypes are readily discernable to the skilled artisan in view of the instant disclosure and, accordingly, are within the scope of the instant disclosure. All immunoglobulin classes are clearly within the scope of the present disclosure, the following discussion will generally be directed to the IgG class of immunoglobulin molecules. With regard to IgG, a standard immunoglobulin molecule comprises two identical light chain polypeptides of molecular weight approximately 23,000 Daltons, and two identical heavy chain polypeptides of molecular weight 53,000-70,000. The four chains are typically joined by disulfide bonds in a “Y” configuration wherein the light chains bracket the heavy chains starting at the mouth of the “Y” and continuing through the variable region.

Antibodies, antigen-binding polypeptides, variants, or derivatives thereof of the disclosure include, but are not limited to, polyclonal, monoclonal, multispecific, human, humanized, primatized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a VK or VH domain, fragments produced by a Fab expression library, and anti-idiotypic (anti-Id) antibodies (including, e.g., anti-Id antibodies to LIGHT antibodies disclosed herein). Immunoglobulin or antibody molecules of the disclosure can be of any type (e.g., IgG, IgE, IgM, IgD, IgA, and IgY), class (e.g., IgG1, IgG2, IgG3, IgG4, IgA1 and IgA2) or subclass of immunoglobulin molecule.

Light chains are classified as either kappa or lambda (K, λ). Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. In this regard, it will be appreciated that the variable domains of both the light (VK) and heavy (VH) chain portions determine antigen recognition and specificity. Conversely, the constant domains of the light chain (CK) and the heavy chain (CH1, CH2 or CH3) confer important biological properties such as secretion, transplacental mobility, Fc receptor binding, complement binding, and the like. By convention the numbering of the constant region domains increases as they become more distal from the antigen-binding site or amino-terminus of the antibody. The N-terminal portion is a variable region and at the C-terminal portion is a constant region; the CH3 and CK domains actually comprise the carboxy-terminus of the heavy and light chain, respectively.

As indicated above, the variable region allows the antibody to selectively recognize and specifically bind epitopes on antigens. That is, the VK domain and VH domain, or subset of the complementarity determining regions (CDRs), of an antibody combine to form the variable region that defines a three dimensional antigen-binding site. This quaternary antibody structure forms the antigen-binding site present at the end of each arm of the Y. More specifically, the antigen-binding site is defined by three CDRs on each of the VH and VK chains (i.e. CDR-H1, CDR-H2, CDR-H3, CDR-L1, CDR-L2 and CDR-L3). In some instances, e.g., certain immunoglobulin molecules derived from camelid species or engineered based on camelid immunoglobulins, a complete immunoglobulin molecule may consist of heavy chains only, with no light chains. See, e.g., Hamers-Casterman et al., Nature 363:446-448 (1993).

In naturally occurring antibodies, the six “complementarity determining regions” or “CDRs” present in each antigen-binding domain are short, non-contiguous sequences of amino acids that are specifically positioned to form the antigen-binding domain as the antibody assumes its three dimensional configuration in an aqueous environment. The remainder of the amino acids in the antigen-binding domains, referred to as “framework” regions, show less inter-molecular variability. The framework regions largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions. The antigen-binding domain formed by the positioned CDRs defines a surface complementary to the epitope on the immunoreactive antigen. This complementary surface promotes the non-covalent binding of the antibody to its cognate epitope. The amino acids comprising the CDRs and the framework regions, respectively, can be readily identified for any given heavy or light chain variable region by one of ordinary skill in the art, since they have been precisely defined (see “Sequences of Proteins of Immunological Interest,” Kabat, E., et al., U.S. Department of Health and Human Services, (1983); and Chothia and Lesk, J. MoI. Biol., 196:901-917 (1987)).

In the case where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. This particular region has been described by Kabat et al., U.S. Dept. of Health and Human Services, “Sequences of Proteins of Immunological Interest” (1983) and by Chothia et al., J. MoI. Biol. 196:901-917 (1987), which are incorporated herein by reference in their entireties. The CDR definitions according to Kabat and Chothia include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth in the table below as a comparison. The exact residue numbers which encompass a particular CDR will vary depending on the sequence and size of the CDR. Those skilled in the art can routinely determine which residues comprise a particular CDR given the variable region amino acid sequence of the antibody.

| | Kabat | Chothia |
|---|---|---|
| CDR-H1 | 31-35 | 26-32 |
| CDR-H2 | 50-65 | 52-58 |
| CDR-H3 | 95-102 | 95-102 |
| CDR-L1 | 24-34 | 26-32 |
| CDR-L2 | 50-56 | 50-52 |
| CDR-L3 | 89-97 | 91-96 |

Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983).

In addition to table above, the Kabat number system describes the CDR regions as follows: CDR-H1 begins at approximately amino acid 31 (i.e., approximately 9 residues after the first cysteine residue), includes approximately 5-7 amino acids, and ends at the next tryptophan residue. CDR-H2 begins at the fifteenth residue after the end of CDR-H1, includes approximately 16-19 amino acids, and ends at the next arginine or lysine residue. CDR-H3 begins at approximately the thirty third amino acid residue after the end of CDR-H2; includes 3-25 amino acids; and ends at the sequence W-G-X-G, where X is any amino acid. CDR-L1 begins at approximately residue 24 (i.e., following a cysteine residue); includes approximately 10-17 residues; and ends at the next tryptophan residue. CDR-L2 begins at approximately the sixteenth residue after the end of CDR-L1 and includes approximately 7 residues. CDR-L3 begins at approximately the thirty third residue after the end of CDR-L2 (i.e., following a cysteine residue); includes approximately 7-11 residues and ends at the sequence F or W-G-X-G, where X is any amino acid.

Antibodies disclosed herein may be from any animal origin including birds and mammals. Preferably, the antibodies are human, murine, donkey, rabbit, goat, guinea pig, camel, llama, horse, or chicken antibodies. In another embodiment, the variable region may be condricthoid in origin (e.g., from sharks).

As used herein, the term “heavy chain constant region” includes amino acid sequences derived from an immunoglobulin heavy chain. A polypeptide comprising a heavy chain constant region comprises at least one of: a CH1 domain, a hinge (e.g., upper, middle, and/or lower hinge region) domain, a CH2 domain, a CH3 domain, or a variant or fragment thereof. For example, an antigen-binding polypeptide for use in the disclosure may comprise a polypeptide chain comprising a CH1 domain; a polypeptide chain comprising a CH1 domain, at least a portion of a hinge domain, and a CH2 domain; a polypeptide chain comprising a CH1 domain and a CH3 domain; a polypeptide chain comprising a CH1 domain, at least a portion of a hinge domain, and a CH3 domain, or a polypeptide chain comprising a CH1 domain, at least a portion of a hinge domain, a CH2 domain, and a CH3 domain. In another embodiment, a polypeptide of the disclosure comprises a polypeptide chain comprising a CH3 domain. Further, an antibody for use in the disclosure may lack at least a portion of a CH2 domain (e.g., all or part of a CH2 domain). As set forth above, it will be understood by one of ordinary skill in the art that the heavy chain constant region may be modified such that they vary in amino acid sequence from the naturally occurring immunoglobulin molecule.

The heavy chain constant region of an antibody disclosed herein may be derived from different immunoglobulin molecules. For example, a heavy chain constant region of a polypeptide may comprise a CH1 domain derived from an IgG₁ molecule and a hinge region derived from an IgG₃ molecule. In another example, a heavy chain constant region can comprise a hinge region derived, in part, from an IgG₁ molecule and, in part, from an IgG₃ molecule. In another example, a heavy chain portion can comprise a chimeric hinge derived, in part, from an IgG₁ molecule and, in part, from an IgG₄ molecule.

As used herein, the term “light chain constant region” includes amino acid sequences derived from antibody light chain. Preferably, the light chain constant region comprises at least one of a constant kappa domain or constant lambda domain.

A “light chain-heavy chain pair” refers to the collection of a light chain and heavy chain that can form a dimer through a disulfide bond between the CL domain of the light chain and the CH1 domain of the heavy chain.

As previously indicated, the subunit structures and three dimensional configuration of the constant regions of the various immunoglobulin classes are well known. As used herein, the term “VH domain” includes the amino terminal variable domain of an immunoglobulin heavy chain and the term “CH1 domain” includes the first (most amino terminal) constant region domain of an immunoglobulin heavy chain. The CH1 domain is adjacent to the VH domain and is amino terminal to the hinge region of an immunoglobulin heavy chain molecule.

As used herein the term “CH2 domain” includes the portion of a heavy chain molecule that extends, e.g., from about residue 244 to residue 360 of an antibody using conventional numbering schemes (residues 244 to 360, Kabat numbering system; and residues 231-340, EU numbering system; see Kabat et al., U.S. Dept. of Health and Human Services, “Sequences of Proteins of Immunological Interest” (1983). The CH2 domain is unique in that it is not closely paired with another domain. Rather, two N-linked branched carbohydrate chains are interposed between the two CH2 domains of an intact native IgG molecule. It is also well documented that the CH3 domain extends from the CH2 domain to the C-terminal of the IgG molecule and comprises approximately 108 residues.

As used herein, the term “hinge region” includes the portion of a heavy chain molecule that joins the CH1 domain to the CH2 domain. This hinge region comprises approximately 25 residues and is flexible, thus allowing the two N-terminal antigen-binding regions to move independently. Hinge regions can be subdivided into three distinct domains: upper, middle, and lower hinge domains (Roux et al., J. Immunol 161:4083 (1998)).

As used herein the term “disulfide bond” includes the covalent bond formed between two sulfur atoms. The amino acid cysteine comprises a thiol group that can form a disulfide bond or bridge with a second thiol group. In most naturally occurring IgG molecules, the CH1 and CK regions are linked by a disulfide bond and the two heavy chains are linked by two disulfide bonds at positions corresponding to 239 and 242 using the Kabat numbering system (position 226 or 229, EU numbering system).

As used herein, the term “chimeric antibody” will be held to mean any antibody wherein the immunoreactive region or site is obtained or derived from a first species and the constant region (which may be intact, partial or modified in accordance with the instant disclosure) is obtained from a second species. In certain embodiments the target binding region or site will be from a non-human source (e.g. mouse or primate) and the constant region is human.

As used herein, “percent humanization” is calculated by determining the number of framework amino acid differences (i.e., non-CDR difference) between the humanized domain and the germline domain, subtracting that number from the total number of amino acids, and then dividing that by the total number of amino acids and multiplying by 100.

By “specifically binds” or “has specificity to,” it is generally meant that an antibody binds to an epitope via its antigen-binding domain, and that the binding entails some complementarity between the antigen-binding domain and the epitope. According to this definition, an antibody is said to “specifically bind” to an epitope when it binds to that epitope, via its antigen-binding domain more readily than it would bind to a random, unrelated epitope. The term “specificity” is used herein to qualify the relative affinity by which a certain antibody binds to a certain epitope. For example, antibody “A” may be deemed to have a higher specificity for a given epitope than antibody “B,” or antibody “A” may be said to bind to epitope “C” with a higher specificity than it has for related epitope “D.”

As used herein, the terms “treat” or “treatment” refer to both therapeutic treatment and prophylactic or preventative measures, wherein the object is to prevent or slow down (lessen) an undesired physiological change or disorder, such as the progression of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sport, or pet animals such as dogs, cats, guinea pigs, rabbits, rats, mice, horses, cattle, cows, and so on.

As used herein, phrases such as “to a patient in need of treatment” or “a subject in need of treatment” includes subjects, such as mammalian subjects, that would benefit from administration of an antibody or composition of the present disclosure used, e.g., for detection, for a diagnostic procedure and/or for treatment.

### Bifunctional Molecules

The present disclosure provides bifunctional molecules that combine specificity to a tumor antigen or immune checkpoint molecule with an IL-7 cytokine activity. Further, it was discovered that, among all the bifunctional formats tested, Format 2 was superior, including having higher stability, to the other tested formats.

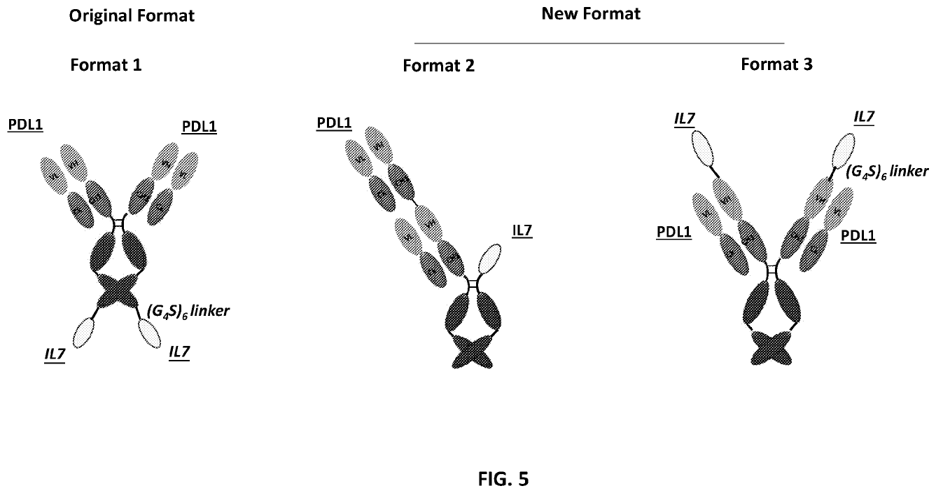

As illustrated in FIG. 5, Format 1 is a symmetrical molecule which includes two copies of a heavy chain and two copies of a light chain. The N-terminal portion is similar to a conventional antibody. Two IL-7 proteins are fused to the C-terminal end of each of the heavy chains.

Format 2, by contrast, is asymmetrical, and includes a longer heavy chain that includes two copies of the VH/CH1 combination and a copy of the conventional Fc fragment. The shorter heavy has an IL-7 protein fused to the N-terminal end of a Fc fragments. In addition, two light chains are paired to the longer heavy chain.

Format 3, like Format 1, is also symmetrical and includes two heavy chains and two light chains. Different from Format 1, the IL-7 proteins are fused to the N-terminal end of the heavy chains. Despite having only copy of the IL-7 protein, Format 2 is not only more stable than Format 1 and Format 2, it also retains excellent IL-7 activity.

In accordance with one embodiment of the present disclosure, therefore, provided is a polypeptide having an antigen-binding unit having specificity to a tumor antigen or immune checkpoint molecule, and an IL-7 protein or homologue, both of which are fused to, preferably the N-terminus, of a Fc fragment.

In some embodiments, provided is a polypeptide comprising: a first portion comprising, in an N-terminal to C-terminal order, a first fragment, a CH2 fragment, and a CH3 fragment, wherein the first fragment comprises an IL-7 protein, an IL-7 protein homologue having at least 75% sequence identity to the IL-7 protein, or a fragment thereof, and wherein the first fragment is capable of binding an IL-7 receptor; and a second portion comprising, in an N-terminal to C-terminal order, an antigen-binding fragment, a CH2 fragment, and a CH3 fragment, wherein the antigen-binding fragment is capable of specifically binding to tumor antigen or an immune checkpoint molecule, wherein the first portion is paired to the second portion through interaction between the CH2 fragments and/or between the CH3 fragments.

The IL-7 protein is known, with a protein sequence deposited in GenBank at accession no. NP_000871.1, of which the mature sequence is provided in SEQ ID NO: 7. Through calculation and testing, it was determined that mutant forms of the IL-7 protein with reduced IL-7 activity maintained synergism with the anti-PD-L1 antibody as compared to the wild-type IL7. Non-limiting examples of such mutants include those having an amino acid substitution at IL7^{W142}. The substitution can be with a non-polar amino acid such as G, A, V, C, P, L, I, M, and F.

The term “IL-7 protein” as used herein refers to the wild-type IL-7, e.g., human IL-7, as well as its biological equivalents, i.e., those that have at least 75%, 80%, 85%, 90%, 95%, 98%, or 99% sequence identity to the wild-type human IL-7 and maintain the activity of the wild-type such as binding to an IL-7 receptor (e.g., receptor alpha), which can be readily measured. In some embodiments, the human IL-7 protein has reduced IL-7 activity as compared to the wild-type. In some embodiments, the reduced IL-7 activity is at least 20%, 30%, 40%, 50%, 60%, 70%, 80% or 90% of binding activity to IL-7 receptor as compared to the wild-type IL-7. In some embodiments, the IL-7 activity is at least 1%, 2%, 5%, 10%, 15%, 20%, 30%, 40%, 50%, 60%, 70%, 80%, 90%, 95%, 97%, 98%, 98%, 99%, 99.9%, 99.99%, 99.999%, 99.9999% lower than that of the wild-type IL-7. In some embodiments, the IL-7 activity is between that of IL-7^{W142A} and wild-type IL-7. In some embodiments, the IL-7 protein is a synthetic analog that is capable of binding to an IL-7 receptor.

A biological equivalent of IL-7 may have increased or reduced binding affinity to IL-7 receptor alpha, increased or reduced stability, increased or reduced IL-7 activity, increased or reduced IL-7 signaling, or reduced immunogenicity, as compared to the wild-type IL-7 protein.

In some embodiments, the human IL-7 protein includes a mutation at W142 as compared to the wild-type. In some embodiments, the mutation is to a non-polar amino acid. Non-limiting examples of the mutation include a mutation to Ala, Gly, Cys, Leu, Ile, Met, Phe, or Val. In some embodiments, the mutation is to Phe, Met, Ile, Leu, Val, or Ala. In one embodiment, the mutation is W142A (e.g., SEQ ID NO: 10). In one embodiment, the mutation is W142I (e.g., SEQ ID NO: 8). In one embodiment, the mutation is W142V (e.g., SEQ ID NO: 9).

A fragment of the IL-7 protein can also be used, in some embodiments. The fragment, in some embodiments, is capable of binding an IL-7 receptor (e.g., receptor alpha), preferably with reduced IL-7 activity as compared to the wild-type protein. The 3-dimensional structures of IL-7 in complex with IL-7 receptors have been demonstrated. See, e.g., McElroy et al., Structure. 2009 Jan. 14; 17(1):54-65. IL-7 adopts an up-up-down-down 4-helix bundle topology with two crossover loops. The α-helices A-D vary in length from 13 to 22 residues. In some embodiments, the fragment includes at least one, two, or three of the alpha helices. In some embodiments, the fragment includes all four of the alpha helices. In some embodiments, the fragment retains interface amino acid residues including S19, D74 and K81.

The IL-7 protein can allow further modifications, such as addition, deletion and/or substitutions, at other amino acid locations as well. Such modifications can be substitution at one, two or three positions. In one embodiment, the modification is substitution at one of the positions. Such substitutions, in some embodiments, are conservative substitutions.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in an immunoglobulin polypeptide is preferably replaced with another amino acid residue from the same side chain family. In another embodiment, a string of amino acids can be replaced with a structurally similar string that differs in order and/or composition of side chain family members.

Non-limiting examples of conservative amino acid substitutions are provided in the table below, where a similarity score of 0 or higher indicates conservative substitution between the two amino acids.

TABLE A
Amino Acid Similarity Matrix

| | C | G | P | S | A | T | D | E | N | Q | H | K | R | V | M | I | L | F | Y | W |
|---|---|---|---|---|---|---|---|---|---|---|---|---|---|---|---|---|---|---|---|---|
| W | −8 | −7 | −6 | −2 | −6 | −5 | −7 | −7 | −4 | −5 | −3 | −3 | 2 | −6 | −4 | −5 | −2 | 0 | 0 | 17 |
| Y | 0 | −5 | −5 | −3 | −3 | −3 | −4 | −4 | −2 | −4 | 0 | −4 | −5 | −2 | −2 | −1 | −1 | 7 | 10 | |
| F | −4 | −5 | −5 | −3 | −4 | −3 | −6 | −5 | −4 | −5 | −2 | −5 | −4 | −1 | 0 | 1 | 2 | 9 | | |
| L | −6 | −4 | −3 | −3 | −2 | −2 | −4 | −3 | −3 | −2 | −2 | −3 | −3 | 2 | 4 | 2 | 6 | | | |
| I | −2 | −3 | −2 | −1 | −1 | 0 | −2 | −2 | −2 | −2 | −2 | −2 | −2 | 4 | 2 | 5 | | | | |
| M | −5 | −3 | −2 | −2 | −1 | −1 | −3 | −2 | 0 | −1 | −2 | 0 | 0 | 2 | 6 | | | | | |
| V | −2 | −1 | −1 | −1 | 0 | 0 | −2 | −2 | −2 | −2 | −2 | −2 | −2 | 4 | | | | | | |
| R | −4 | −3 | 0 | 0 | −2 | −1 | −1 | −1 | 0 | 1 | 2 | 3 | 6 | | | | | | | |
| K | −5 | −2 | −1 | 0 | −1 | 0 | 0 | 0 | 1 | 1 | 0 | 5 | | | | | | | | |
| H | −3 | −2 | 0 | −1 | −1 | −1 | 1 | 1 | 2 | 3 | 6 | | | | | | | | | |
| Q | −5 | −1 | 0 | −1 | 0 | −1 | 2 | 2 | 1 | 4 | | | | | | | | | | |
| N | −4 | 0 | −1 | 1 | 0 | 0 | 2 | 1 | 2 | | | | | | | | | | | |
| E | −5 | 0 | −1 | 0 | 0 | 0 | 3 | 4 | | | | | | | | | | | | |
| D | −5 | 1 | −1 | 0 | 0 | 0 | 4 | | | | | | | | | | | | | |
| T | −2 | 0 | 0 | 1 | 1 | 3 | | | | | | | | | | | | | | |
| A | −2 | 1 | 1 | 1 | 2 | | | | | | | | | | | | | | | |
| S | 0 | 1 | 1 | 1 | | | | | | | | | | | | | | | | |
| P | −3 | −1 | 6 | | | | | | | | | | | | | | | | | |
| G | −3 | 5 | | | | | | | | | | | | | | | | | | |
| C | 12 | | | | | | | | | | | | | | | | | | | |

TABLE B
Conservative Amino Acid Substitutions

| For Amino Acid | Substitution With |
|---|---|
| Alanine | D-Ala, Gly, Aib, β-Ala, L-Cys, D-Cys |
| Arginine | D-Arg, Lys, D-Lys, Orn D-Orn |
| Asparagine | D-Asn, Asp, D-Asp, Glu, D-Glu Gln, D-Gln |
| Aspartic Acid | D-Asp, D-Asn, Asn, Glu, D-Glu, Gln, D-Gln |
| Cysteine | D-Cys, S-Me-Cys, Met, D-Met, Thr, D-Thr, L-Ser, D-Ser |
| Glutamine | D-Gln, Asn, D-Asn, Glu, D-Glu, Asp, D-Asp |
| Glutamic Acid | D-Glu, D-Asp, Asp, Asn, D-Asn, Gln, D-Gln |
| Glycine | Ala, D-Ala, Pro, D-Pro, Aib, β-Ala |
| Isoleucine | D-Ile, Val, D-Val, Leu, D-Leu, Met, D-Met |
| Leucine | Val, D-Val, Met, D-Met, D-Ile, D-Leu, Ile |
| Lysine | D-Lys, Arg, D-Arg, Orn, D-Orn |
| Methionine | D-Met, S-Me-Cys, Ile, D-Ile, Leu, D-Leu, Val, D-Val |
| Phenylalanine | D-Phe, Tyr, D-Tyr, His, D-His, Trp, D-Trp |
| Proline | D-Pro |
| Serine | D-Ser, Thr, D-Thr, allo-Thr, L-Cys, D-Cys |
| Threonine | D-Thr, Ser, D-Ser, allo-Thr, Met, D-Met, Val, D-Val |
| Tyrosine | D-Tyr, Phe, D-Phe, His, D-His, Trp, D-Trp |
| Valine | D-Val, Leu, D-Leu, Ile, D-Ile, Met, D-Met |

The antigen-binding fragment may include a Fab fragment, a single-chain variable fragment (scFv), a nanobody, an antigen-binding motif or a combination thereof. In some embodiments, the antigen-binding fragment has at least two binding sites, such as including two Fab, two scFv, two nanobodies, or a combination of Fab, scFv, or nanobody.

An abundance of tumor antigens are known in the art and new tumor antigens can be readily identified by screening. Non-limiting examples of tumor antigens include EGFR, Her2, EpCAM, CD20, CD30, CD33, CD47, CD52, CD133, CD73, CEA, gpA33, Mucins, TAG-72, CIX, PSMA, folate-binding protein, GD2, GD3, GM2, VEGF, VEGFR, Integrin, αVβ3, α5β1, ERBB2, ERBB3, MET, IGF1R, EPHA3, TRAILR1, TRAILR2, RANKL, FAP and Tenascin.

Immune checkpoints are molecules in the immune system that either turn up a signal (co-stimulatory molecules) or turn down a signal (co-inhibitory molecules). Many cancers protect themselves from the immune system by inhibiting the T cell signal through agonist for co-inhibitory molecules or antagonist for co-stimulatory molecules. Non-limiting examples include PD-L1, PD-1, CTLA-4, LAG-3 (also known as CD223), CD28, CD122, 4-1BB (also known as CD137), TIM3, OX-40/OX40L, CD40/CD40L, LIGHT, ICOS/ICOSL, GITR/GITRL, TIGIT, CD27, VISTA, B7H3, B7H4, HEVM or BTLA (also known as CD272).

Programmed death-ligand 1 (PD-L1), also known as cluster of differentiation 274 (CD274) or B7 homolog 1 (B7-H1), is a 40 kDa type 1 transmembrane protein believed to play a major role in suppressing the immune system during particular events such as pregnancy, tissue allografts, autoimmune disease and other disease states such as hepatitis. The binding of PD-L1 to PD-1 or B7.1 transmits an inhibitory signal which reduces the proliferation of CD8+ T cells at the lymph nodes and supplementary to that PD-1 is also able to control the accumulation of foreign antigen specific T cells in the lymph nodes through apoptosis which is further mediated by a lower regulation of the gene Bcl-2.

It has been shown that upregulation of PD-L1 may allow cancers to evade the host immune system. An analysis of tumor specimens from patients with renal cell carcinoma found that high tumor expression of PD-L1 was associated with increased tumor aggressiveness and an increased risk of death. Many PD-L1 inhibitors are in development as immuno-oncology therapies and are showing good results in clinical trials.

In addition to treatment of cancers, PD-L1 inhibition has also shown promises in treating infectious diseases. In a mouse model of intracellular infection, L. monocytogenes induced PD-L1 protein expression in T cells, NK cells, and macrophages. PD-L1 blockade (e.g., using blocking antibodies) resulted in increased mortality for infected mice. Blockade reduced TNFα and nitric oxide production by macrophages, reduced granzyme B production by NK cells, and decreased proliferation of L. monocytogenes antigen-specific CD8 T cells (but not CD4 T cells). This evidence suggests that PD-L1 acts as a positive costimulatory molecule in intracellular infection.

Non-limiting examples of anti-PD-L1 antibodies and their respective fragments are disclosed and tested here. In some embodiments, an anti-PD-L1 antibody or fragment that can be used here includes a VH that comprises a CDR1, CDR2, and CDR3 having the amino acid sequences of residues 31-35, residues 50-66, and residues 99-108 of SEQ ID NO:1. In some embodiments, an anti-PD-L1 antibody or fragment that can be used here includes a VH that comprises a CDR1, CDR2, and CDR3 having the amino acid sequences of residues 31-35, residues 50-66, and residues 99-108 of SEQ ID NO:3. In some embodiments, an anti-PD-L1 antibody or fragment that can be used here includes a VH that comprises a CDR1, CDR2, and CDR3 having the amino acid sequences of residues 31-35, residues 50-66, and residues 99-108 of SEQ ID NO:4. In some embodiments, an anti-PD-L1 antibody or fragment that can be used here includes a VH that comprises a CDR1, CDR2, and CDR3 having the amino acid sequences of residues 31-35, residues 50-66, and residues 99-108 of SEQ ID NO:5. Example VH sequences include SEQ ID NO:1, 3, 4, and 5.

In some embodiments, an anti-PD-L1 antibody or fragment that can be used here includes a VL that comprises a CDR1, CDR2, and CDR3 having the amino acid sequences of residues 24-34, residues 50-56, and residues 89-97 of SEQ ID NO:2. In some embodiments, an anti-PD-L1 antibody or fragment that can be used here includes a VL that comprises a CDR1, CDR2, and CDR3 having the amino acid sequences of residues 24-34, residues 50-56, and residues 89-97 of SEQ ID NO:6. Example VL sequences include SEQ ID NO:2 and 6.

In some embodiments, the first chain comprises the amino acid sequence of SEQ ID NO:27 or 31. In some embodiments, the second chain comprises the amino acid sequence of SEQ ID NO:34, 35 or 36. In some embodiments, the third chain and fourth chain each comprises the amino acid sequence of SEQ ID NO:25 or 33.

In an example embodiment of the present disclosure, provided is a polypeptide comprising: a first chain comprising, in an N-terminal to C-terminal order, a first fragment, a CH2 fragment, and a CH3 fragment, wherein the first fragment comprises an IL-7 protein, an IL-7 homologue having at least 75% sequence identity to the IL-7 protein, or a fragment thereof, and wherein the first fragment is capable of binding an IL-7 receptor; a second chain comprising, in an N-terminal to C-terminal order, a first heavy chain variable region (VH), a first heavy chain constant region (CH1), a second heavy chain variable region (VH), a second heavy chain constant region (CH1), a CH2 fragment and a CH3 fragment; a third chain comprising, in an N-terminal to C-terminal order, a light chain variable region (VL) and a light chain constant region (CL); and a fourth chain comprising, in an N-terminal to C-terminal order, a light chain variable region (VL) and a light chain constant region (CL). In some embodiments, the CH2 fragment and CH3 fragment of the first chain are paired with the CH2 fragment and the CH3 fragment of the second chain. In some embodiments, the first VH of the second chain is paired with the VL of the third chain which, collectively, are capable of binding a human PD-L1 protein. In some embodiments, the second VH of the second chain is paired with the VL of the fourth chain which, collectively, are capable of binding a human PD-L1 protein.

In some embodiments, linkers or hingers are included between some of the neighboring domains or fragments. For instance, a hinge region can be included in the first chain (the longer heavy chain) between the two VH/CH1 units. The hinge region can include a sequence such as SEQ ID NO:17 and 18. In another example, a hinge region can be included in the heavy chain between the second VH/CH1 unit and the Fc fragment. The hinge region can include a sequence such as SEQ ID NO:19 and 20 on the second chain (the shorter heavy chain) or SEQ ID NO:17 or 18 on the first chain (the longer heavy chain). SEQ ID NO:19 and 20 do not include a Cysteine. The removal of the Cysteine here can be helpful as the shorter heavy chain, which is fused to an IL-7 protein, does not need to pair with a light chain; hence such a hinge can avoid its pairing to a light chain.

Peptide linkers can also be optionally used. The peptide linker is generally a peptide that has from 5 to 100 amino acid residues. Preferably, the linker includes enough smaller amino acids to ensure its flexibility. For instance, the length of the linker can be from 5 to 100 amino acids, from 10 to 90 amino acids, from 10 to 80 amino acids, from 10 to 75 amino acids, from 15 to 90 amino acids, from 15 to 80 amino acids, from 15 to 70 amino acids, from 20 to 80 amino acids, from 20 to 70 amino acids, from 20 to 60 amino acids, from 25 to 90 amino acids, from 25 to 80 amino acids, from 25 to 75 amino acids, from 25 to 70 amino acids, from 25 to 60 amino acids, from 30 to 80 amino acids, from 30 to 70 amino acids, from 30 to 60 amino acids, or from 40 to 70 amino acids, without limitation.

The flexibility of the linker can be achieved by incorporating a minimum percentage of smaller amino acids, e.g., alanine, glycine, cysteine, and serine. In some embodiment, the linker includes at least 10%, 15%, 20%, 25%, 30%, 35%, 40%, 45%, 50%, 55%, 60%, 65%, 70%, 75% or 80% of amino acids selected from alanine, glycine, cysteine, or serine. Non-limiting examples of peptide linkers are provided in SEQ ID NO:11.

| | | SEQ ID |
|---|---|---|
| Name | Sequence | NO: |
| Anti-PD-L1 v1 | EVQLVESGGGLVQPGGSLRLSCAASGFTFSSYDMSWVRQAPGKSLEWVATISDAGGY | 1 |
| VH | IYYRDSVKGRFTISRDNAKNSLYLQMNSLRDEDTAVYICARELPWRYALDYWGQGTT | |
| | VTVSS | |
| Anti-PD-L1 | DIQMTQSPSSLSASVGDRVTITCKASQDVTPAVAWYQQKPGKAPKLLIYSTSSRYTG | 2 |
| v1/v2/v3 VL | VPSRFSGSGSGTDFTFTISSLQPEDIATYYCQQHYTTPLTFGQGTKLEIK | |
| Anti-PD-L1 v2 | EVQLVESGGGLVQPGGSLRLSCAASGFTFSSYDMSWVRQAPGKSLEWVATISDAGGY | 3 |
| VH | IYYRDSVKGRFTISRDNAKNSLYLQMNSLRDEDTAVYICAREFGKRYALDYWGQGTT | |
| | VTVSS | |
| Anti-PD-L1 v3 | EVQLVESGGGLVQPGGSLRLSCAASGFTFSSYDMSWVRQAPGKSLEWVATISDAGGY | 4 |
| VH | IYYSDSVKGRFTISRDNAKNSLYLQMNSLRDEDTAVYICAREFGKRYALDYWGQGTT | |
| | VTVSS | |
| Anti-PD-L1 v4 | QVQLLESGGGLVQPGGSLRLSCAASGFTFSSYWMSWVRQAPGKGLEWVANIKQDGSE | 5 |
| VH | KYYVDSVKGRFTISRDNAKNSLYLQMNSLRAEDTAVYYCARVALWDDAFDIWGQGTM | |
| | VTVSS | |
| Anti-PD-L1 v4 | DIQMTQSPSTLSASVGDRVIITCRASRGISSWLAWYQQKPGKAPNLLISKASSLESG | 6 |
| VL | VPSRFSGSGSGTDFTLTISSLQPEDFATYYCQQSSSIPLTFGGGTKVEIK | |
| IL7 WT | DCDIEGKDGKQYESVLMVSIDQLLDSMKEIGSNCLNNEFNFFKRHICDANKEGMFLF | 7 |
| | RAARKLRQFLKMNSTGDFDLHLLKVSEGTTILLNCTGQVKGRKPAALGEAQPTKSLE | |
| | ENKSLKEQKKLNDLCFLKRLLQEIKTCWNKILMGTKEH | |
| IL7 142I | DCDIEGKDGKQYESVLMVSIDQLLDSMKEIGSNCLNNEFNFFKRHICDANKEGMFLF | 8 |
| | RAARKLRQFLKMNSTGDFDLHLLKVSEGTTILLNCTGQVKGRKPAALGEAQPTKSLE | |
| | ENKSLKEQKKLNDLCFLKRLLQEIKTCINKILMGTKEH | |
| IL7 142V | DCDIEGKDGKQYESVLMVSIDQLLDSMKEIGSNCLNNEFNFFKRHICDANKEGMFLF | 9 |
| | RAARKLRQFLKMNSTGDFDLHLLKVSEGTTILLNCTGQVKGRKPAALGEAQPTKSLE | |
| | ENKSLKEQKKLNDLCFLKRLLQEIKTCVNKILMGTKEH | |
| IL7 142A | DCDIEGKDGKQYESVLMVSIDQLLDSMKEIGSNCLNNEFNFFKRHICDANKEGMFLF | 10 |
| | RAARKLRQFLKMNSTGDFDLHLLKVSEGTTILLNCTGQVKGRKPAALGEAQPTKSLE | |
| | ENKSLKEQKKLNDLCFLKRLLQEIKTCANKILMGTKEH | |
| G4S linker | GGGGSGGGGSGGGGSGGGGSGGGGSGGGGS | 11 |
| CL | RTVAAPSVFIFPPSDEQLKSGTASVVCLLNNFYPREAKVQWKVDNALQSGNSQESVT | 12 |
| | EQDSKDSTYSLSSTLTLSKADYEKHKVYACEVTHQGLSSPVTKSFNRGEC | |
| CH1 | ASTKGPSVFPLAPSSKSTSGGTAALGCLVKDYFPEPVTVSWNSGALTSGVHTFPAVL | 13 |
| | QSSGLYSLSSVVTVPSSSLGTQTYICNVNHKPSNTKVDKKV | |
| Fc (CH2-CH3) | APELLGGPSVFLFPPKPKDTLMISRTPEVTCVVVDVSHEDPEVKFNWYVDGVEVHNA | 14 |
| v1 | KTKPREEQYNSTYRVVSVLTVLHQDWLNGKEYKCKVSNKALPAPIEKTISKAKGQPR | |
| | EPQVYTLPPSREEMTKNQVSLTCLVKGFYPSDIAVEWESNGQPENNYKTTPPVLDSD | |
| | GSFFLYSKLTVDKSRWQQGNVFSCSVMHEALHNHYTQKSLSLSPGK | |
| Fc v2 | APELLGGPSVFLFPPKPKDTLMISRTPEVTCVVVDVSHEDPEVKFNWYVDGVEVHNA | 15 |
| | KTKPREEQYNSTYRVVSVLTVLHQDWLNGKEYKCKVSNKALPAPIEKTISKAKGQPR | |
| | EPQVYTLPPSREEMTKNQVSLWCLVKGFYPSDIAVEWESNGQPENNYKTTPPVLDSD | |
| | GSFFLYSKLTVDKSRWQQGNVFSCSVMHEALHNHYTQKSLSLSPGK | |
| Fc v3 | APELLGGPSVFLFPPKPKDTLMISRTPEVTCVVVDVSHEDPEVKFNWYVDGVEVHNA | 16 |
| | KTKPREEQYNSTYRVVSVLTVLHQDWLNGKEYKCKVSNKALPAPIEKTISKAKGQPR | |
| | EPQVYTLPPSREEMTKNQVSLSCAVKGFYPSDIAVEWESNGQPENNYKTTPPVLDSD | |
| | GSFFLVSKLTVDKSRWQQGNVFSCSVMHEALHNHYTQKSLSLSPGK | |
| Hinge v1 | EPKSC | 17 |
| Hinge v2 | EPKSCDKTHTCPPCP | 18 |
| Hinge v3 | EPKSADKTHTCPPCP | 19 |
| Hinge v4 | EPKSA | 20 |

In some embodiments, the anti-PD-L1 antibody or fragment is of an isotype of IgG, IgM, IgA, IgE or IgD, and the fragment can take any form, such as a single-chain fragment, a Fab fragment, or a pair of Fab fragments. In some embodiments, the anti-PD-L1 antibody is ADCC-enabled.

Antibodies, variants, or derivatives thereof of the disclosure include derivatives that are modified, i.e., by the covalent attachment of any type of molecule to the antibody such that covalent attachment does not prevent the antibody from binding to the epitope. For example, but not by way of limitation, the antibodies can be modified, e.g., by glycosylation, acetylation, pegylation, phosphorylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the antibodies may contain one or more non-classical amino acids.

### Polynucleotides Encoding the Polypeptides and Methods of Preparing the Polypeptides

The present disclosure also provides isolated polynucleotides or nucleic acid molecules encoding the decoy PD-1 proteins, antibodies, fusion molecules, variants or derivatives thereof of the disclosure. The polynucleotides of the present disclosure may encode the entire heavy and light chain variable regions of the antigen-binding polypeptides, variants or derivatives thereof on the same polynucleotide molecule or on separate polynucleotide molecules. Additionally, the polynucleotides of the present disclosure may encode portions of the heavy and light chain variable regions of the antigen-binding polypeptides, variants or derivatives thereof on the same polynucleotide molecule or on separate polynucleotide molecules.

Methods of making decoy proteins and antibodies are well known in the art and described herein. In certain embodiments, both the variable and constant regions of the antigen-binding polypeptides of the present disclosure are fully human. Fully human antibodies can be made using techniques described in the art and as described herein. For example, fully human antibodies against a specific antigen can be prepared by administering the antigen to a transgenic animal which has been modified to produce such antibodies in response to antigenic challenge, but whose endogenous loci have been disabled. Exemplary techniques that can be used to make such antibodies are described in U.S. Pat. Nos. 6,150,584; 6,458,592; 6,420,140 which are incorporated by reference in their entireties.

### Cancer Treatment

As demonstrated herein, the fusion molecules of the present disclosure exhibited synergistic effects in treating cancer, and may be used in certain treatment and diagnostic methods.

The present disclosure is further directed to therapies which involve administering the fusion molecules of the disclosure to a patient such as an animal, a mammal, and a human for treating one or more of the disorders or conditions described herein. Therapeutic compounds of the disclosure include, but are not limited to, fusion molecules of the disclosure (including variants and derivatives thereof as described herein) and nucleic acids or polynucleotides encoding fusion molecules of the disclosure (including variants and derivatives thereof as described herein).

The therapy can also involve administering IL-7 variants as disclosed herein, optionally in combination with administration of a PD-L1 inhibitor as disclosed herein. In some embodiments, the IL-7 variant administered and the PD-L1 inhibitor administered have a molar ratio that is at least 5:1, 4:1, 3:1, 2:1, 1.5:1, 1:1, or 1:2. In some embodiments, the IL-7 variant administered and the PD-L1 inhibitor administered have a molar ratio that is not greater than 2:1, 1.5:1, 1:1, 1:2, 1:3, 1:4, or 1:5. In some embodiments, the IL-7 variant administered and the PD-L1 inhibitor administered have a molar ratio between 2:1 and 1:2 or between 1.5:1 and 1:1.5.

Cellular therapies, such as chimeric antigen receptor (CAR) T-cell therapies, are also provided in the present disclosure. A suitable cell can be used, that is put in contact with an anti-PD-L1 antibody of the present disclosure (or alternatively engineered to express an anti-PD-L1 antibody of the present disclosure). Upon such contact or engineering, the cell can then be introduced to a cancer patient in need of a treatment. The cancer patient may have a cancer of any of the types as disclosed herein. The cell (e.g., T cell) can be, for instance, a tumor-infiltrating T lymphocyte, a CD4+ T cell, a CD8+ T cell, or the combination thereof, without limitation.

In some embodiments, the cell was isolated from the cancer patient him- or her-self. In some embodiments, the cell was provided by a donor or from a cell bank. When the cell is isolated from the cancer patient, undesired immune reactions can be minimized.

Additional diseases or conditions associated with increased cell survival, that may be treated, prevented, diagnosed and/or prognosed with the fusion molecules or variants, or derivatives thereof of the disclosure include, but are not limited to, progression, and/or metastases of malignancies and related disorders such as leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyo sarcoma, colon carcinoma, pancreatic cancer, breast cancer, thyroid cancer, endometrial cancer, melanoma, prostate cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma and retinoblastoma.

A specific dosage and treatment regimen for any particular patient will depend upon a variety of factors, including the particular fusion molecules, variant or derivative thereof used, the patient's age, body weight, general health, sex, and diet, and the time of administration, rate of excretion, drug combination, and the severity of the particular disease being treated. Judgment of such factors by medical caregivers is within the ordinary skill in the art. The amount will also depend on the individual patient to be treated, the route of administration, the type of formulation, the characteristics of the compound used, the severity of the disease, and the desired effect. The amount used can be determined by pharmacological and pharmacokinetic principles well known in the art.

Methods of administration of the fusion molecules, variants or include but are not limited to intradermal, intramuscular, intraperitoneal, intravenous, subcutaneous, intranasal, epidural, and oral routes. The antigen-binding polypeptides or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Thus, pharmaceutical compositions containing the antigen-binding polypeptides of the disclosure may be administered orally, rectally, parenterally, intracistemally, intravaginally, intraperitoneally, topically (as by powders, ointments, drops or transdermal patch), bucally, or as an oral or nasal spray.

The term “parenteral” as used herein refers to modes of administration which include intravenous, intramuscular, intraperitoneal, intrasternal, subcutaneous and intra-articular injection and infusion.

Administration can be systemic or local. In addition, it may be desirable to introduce the fusion molecules of the disclosure into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

It may be desirable to administer the fusion molecules or compositions of the disclosure locally to the area in need of treatment; this may be achieved by, for example, and not by way of limitation, local infusion during surgery, topical application, e.g., in conjunction, with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an antibody, of the disclosure, care must be taken to use materials to which the protein does not absorb.

The amount of the fusion molecules of the disclosure which will be effective in the treatment, inhibition and prevention of an inflammatory, immune or malignant disease, disorder or condition can be determined by standard clinical techniques. In addition, in vitro assays may optionally be employed to help identify optimal dosage ranges. The precise dose to be employed in the formulation will also depend on the route of administration, and the seriousness of the disease, disorder or condition, and should be decided according to the judgment of the practitioner and each patient's circumstances. Effective doses may be extrapolated from dose-response curves derived from in vitro or animal model test systems.

As a general proposition, the dosage administered to a patient of the antigen-binding polypeptides of the present disclosure is typically 0.1 mg/kg to 100 mg/kg of the patient's body weight, between 0.1 mg/kg and 20 mg/kg of the patient's body weight, or 1 mg/kg to 10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of fusion molecules of the disclosure may be reduced by enhancing uptake and tissue penetration (e.g., into the brain) of the fusion molecules by modifications such as, for example, lipidation.

### Compositions

The present disclosure also provides pharmaceutical compositions. Such compositions comprise an effective amount of a fusion molecule, and an acceptable carrier. In some embodiments, the composition further includes a second anticancer agent (e.g., an immune checkpoint inhibitor).

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. Further, a “pharmaceutically acceptable carrier” will generally be a non-toxic solid, semisolid or liquid filler, diluent, encapsulating material or formulation auxiliary of any type.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose are also envisioned. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences by E. W. Martin, incorporated herein by reference. Such compositions will contain a therapeutically effective amount of the antigen-binding polypeptide, preferably in purified form, together with a suitable amount of carrier so as to provide the form for proper administration to the patient. The formulation should suit the mode of administration. The parental preparation can be enclosed in ampoules, disposable syringes or multiple dose vials made of glass or plastic.

In an embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

### EXAMPLES

### Example 1: In Vivo PK/PD Study of Anti-PDL1-IL7 Variants in Cynomolgus Monkey

This example tested a number of bi-functional anti-PDL1-IL7 molecules that adopted a “Format 1” format, which is illustrated in FIG. 5. The sequences of these molecules are provided in Table 1. In this format, the C terminus of the Fc region of an anti-PDL1 antibody was fused to a human IL7 protein through a linker. These bi-functional molecules combined PDL1 antagonism with IL7 cellular activity.

Naïve cynomolgus monkeys (n=2, 1 male/1 female) were intravenously injected with anti-PDL1-IL7 (ref. as L1I7) wild type (L1I7^{WT}) and mutant molecules (L1I7^{142I}, L1I7^{142V} and L1I7^{142A}) at 20 mg/kg per week for three times. Their blood was collected by venipuncture into tubes with no anticoagulant at different time points. In order to detect the serum level of the whole molecule of L1I7 proteins, serum was measured by ELISA using his-tagged PDL1 protein as the coating reagent, followed by detection with biotinylated anti-IL7 detection antibody and secondary antibody Streptavidin-HRP and finally with color-developing agent TMB. To evaluate the stability of L1I7 protein in vivo, the serum level of the anti-PDL1 part of L1I7 was also detected by ELISA. Briefly, his-tagged PDL1 protein was used as the coating reagent, the HRP-anti-human IgG Fc was used as the detection antibody followed by color development.

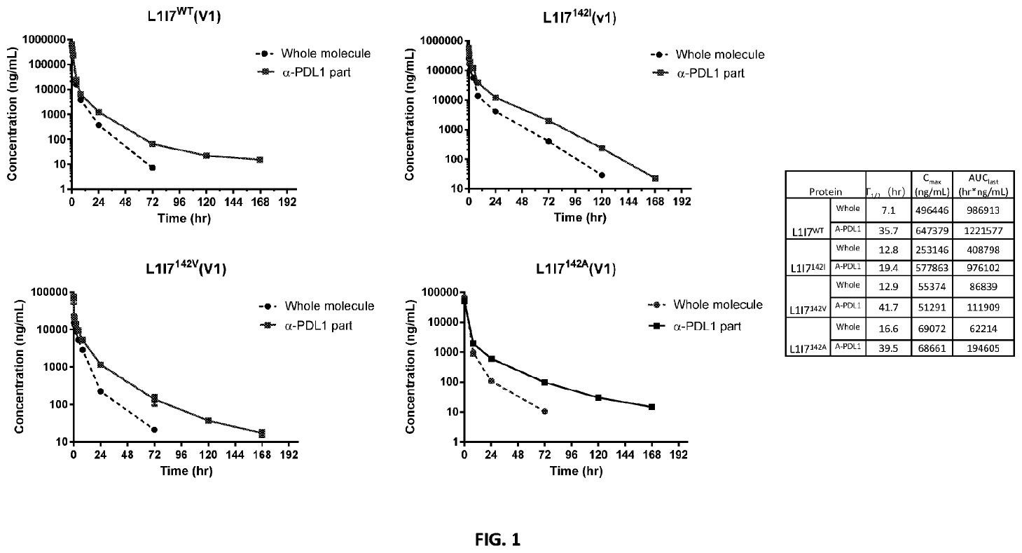

The concentration-time curves of the serum level of different L1I7 molecules after the first dose is shown in FIG. 1. Pharmacokinetic parameters were analyzed by Winolin and shown in FIG. 1. The results showed that the half-life (T_{1/2}) of the whole molecule of all L1I7 proteins was relatively shorter than those of their PDL1 Ab part (e.g., T_{1/2} whole vs. α-PDL1 of L1I7^{WT}: 7.1 vs. 35.7 hours), suggesting breakdown of the anti-PDL1 part from the IL7 part in vivo. In addition, the relatively prolonged T_{1/2} of the whole molecule of the L1I7 molecules with attenuated IL7 activities was observed when compared with L1I7^{WT} (T_{1/2}: L1I7^{142A}>L1I7^{142V}≈L1I7^{142I}>L1I7^{WT}).

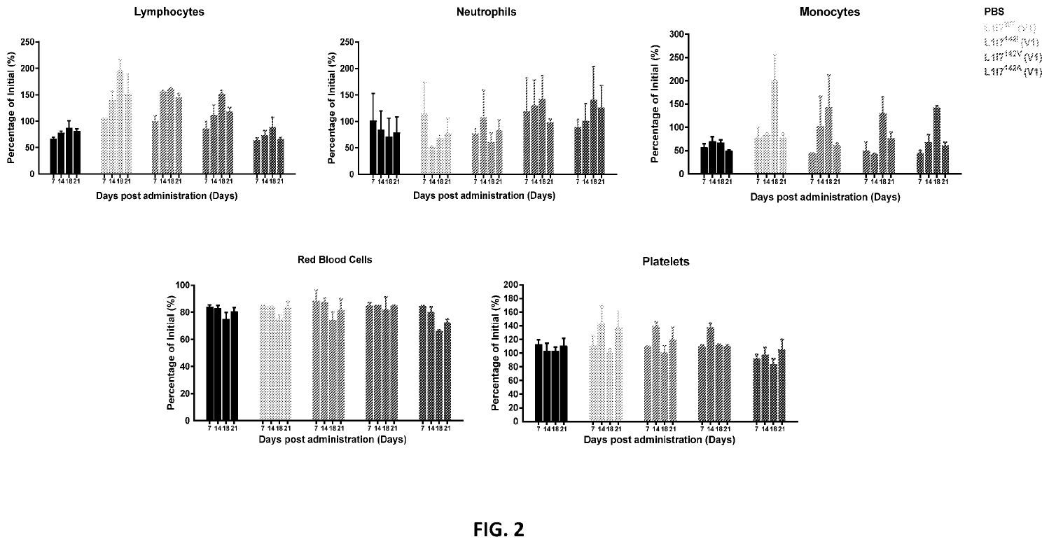

IL7 is a hemostatic cytokine promoting T cell proliferation. To evaluate the in vivo pharmacodynamics of these four L1I7 molecules, the absolute cell counts of individual immune cell types at different time points were analyzed by hematology analyzer ADVIA® 2120 (Siemens). As shown in FIG. 2, the lymphocyte cell counts increased at day 14 (7 days after the second dose), day 18 (4 days after the third dose) and day 21 (7 days after the third dose) in L1I7^{WT} and L1I7^{142I} to a lesser extent. L1I7^{142V} with relative lower IL7 activity compared to L1I7^{142I} showed increased lymphocyte cell counts only at day 18. On the contrary, no change of lymphocyte cell counts was observed in L1I7^{142A} group, which had the weakest IL7 activity compared with other three molecules. Furthermore, no change was observed in the amounts of red blood cells, platelets, neutrophils and monocytes (FIG. 2).

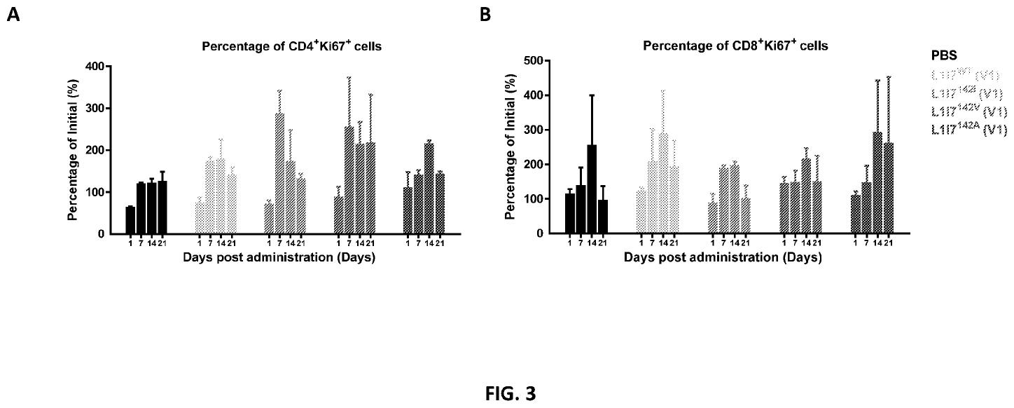

Mechanistically, T cell proliferation measured by Ki67 expression on CD4⁺ or CD8⁺ T cells was promoted by all four L1I7 molecules, indicating the role of IL7 on T cell proliferation promotion in vivo (FIG. 3).

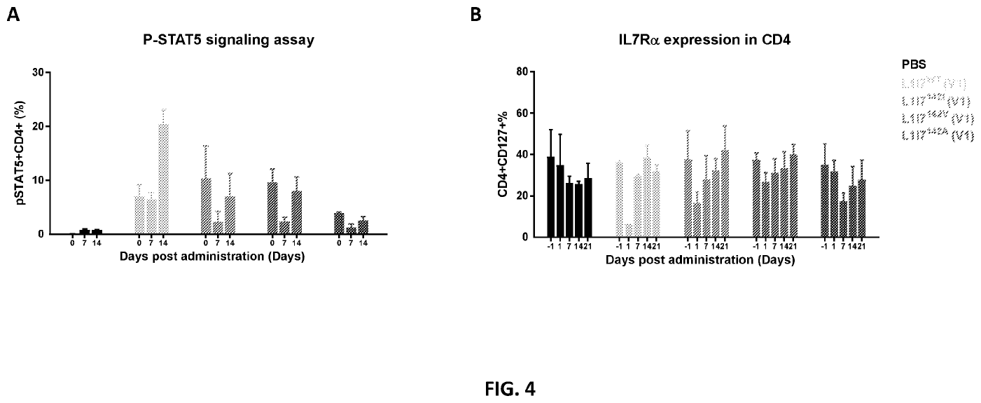

STAT5 phosphorylation (P-STAT5), downstream of IL7 signal on CD4⁺ T cells was detected by FACS at 1 hours after each dose (FIG. 4A). Percentage of P-STAT5⁺CD4⁺ T cells increased at day 0 (first dose) and day 14 (third dose) in all L1I7 molecule treatment groups although the intensity of P-STAT5 activation in L1I7^{142A} group was weakest among these four L1I7 molecules, which was consistent with the unchanged lymphocyte counts in this group.

IL7 receptor α (IL7Rα) is internalized upon conjugating with its ligand IL7. The expression level of IL7Rα is inversely corelated with the binding affinity of IL7 to the receptor. To detect the internalization of IL7Rα on CD4⁺ T cells in each group, the expression level of IL7Rα was detected by FACS at different time points. It was shown that L1I7^{WT} induced significant internalization of IL7Rα at day 1 after first dosing and the expression level of IL7Rα recovered after day 7 (FIG. 4B). Secondary doses did not induce the internalization anymore (FIG. 4B). On the contrary, attenuated L1I7 (L1I7^{142I}, L1I7^{142V} and L1I7^{142A}) induced relatively weaker internalization of IL7Rα compared with L1I7^{WT}.

TABLE 1
Format 1 chains (CDR residues underlined; mutation sites bolded)

| | | SEQ |
|---|---|---|
| Name | Sequence | ID NO: |
| L1I7^{WT} (anti-PD- | EVQLVESGGGLVQPGGSLRLSCAASGFTFSSYDMSWVRQAPGKSLEWVATISDAGGYIYYRD | 21 |
| L1 v1) | SVKGRFTISRDNAKNSLYLQMNSLRDEDTAVYICARELPWRYALDYWGQGTTVTVSS (anti- | |
| Heavy chain | PD-L1 v1 VH fragment; SEQ ID NO: 1) | |
| | ASTKGPSVFPLAPSSKSTSGGTAALGCLVKDYFPEPVTVSWNSGALTSGVHTFPAVLQSSGL | |
| | YSLSSVVTVPSSSLGTQTYICNVNHKPSNTKVDKKV (CH1; SEQ ID NO: 13) | |
| | EPKSCDKTHTCPPCP (Hinge v2; SEQ ID NO: 18) | |
| | APELLGGPSVFLFPPKPKDTLMISRTPEVTCVVVDVSHEDPEVKFNWYVDGVEVHNAKTKPR | |
| | EEQYNSTYRVVSVLTVLHQDWLNGKEYKCKVSNKALPAPIEKTISKAKGQPREPQVYTLPPS | |
| | REEMTKNQVSLTCLVKGFYPSDIAVEWESNGQPENNYKTTPPVLDSDGSFFLYSKLTVDKSR | |
| | WQQGNVFSCSVMHEALHNHYTQKSLSLSPGK (CH2-CH3/Fc v1; SEQ ID NO: 14) | |
| | GGGGSGGGGSGGGGSGGGGSGGGGSGGGGS (G4S linker; SEQ ID NO: 11) | |
| | DCDIEGKDGKQYESVLMVSIDQLLDSMKEIGSNCLNNEFNFFKRHICDANKEGMFLFRAARK | |
| | LRQFLKMNSTGDFDLHLLKVSEGTTILLNCTGQVKGRKPAALGEAQPTKSLEENKSLKEQKK | |
| | LNDLCFLKRLLQEIKTCWNKILMGTKEH (IL7 wild-type; SEQ ID NO: 7) | |
| L1I7^{142I} (anti-pD- | EVQLVESGGGLVQPGGSLRLSCAASGFTFSSYDMSWVRQAPGKSLEWVATISDAGGYIYYRD | 22 |
| L1 v1) Format 1 | SVKGRFTISRDNAKNSLYLQMNSLRDEDTAVYICARELPWRYALDYWGQGTTVTVSS (anti- | |
| Heavy chain | PD-L1 v1 VH fragment; SEQ ID NO: 1) | |
| | ASTKGPSVFPLAPSSKSTSGGTAALGCLVKDYFPEPVTVSWNSGALTSGVHTFPAVLQSSGL | |
| | YSLSSVVTVPSSSLGTQTYICNVNHKPSNTKVDKKV (CH1; SEQ ID NO: 13) | |
| | EPKSCDKTHTCPPCP (Hinge v2; SEQ ID NO: 18) | |
| | APELLGGPSVFLFPPKPKDTLMISRTPEVTCVVVDVSHEDPEVKFNWYVDGVEVHNAKTKPR | |
| | EEQYNSTYRVVSVLTVLHQDWLNGKEYKCKVSNKALPAPIEKTISKAKGQPREPQVYTLPPS | |
| | REEMTKNQVSLTCLVKGFYPSDIAVEWESNGQPENNYKTTPPVLDSDGSFFLYSKLTVDKSR | |
| | WQQGNVFSCSVMHEALHNHYTQKSLSLSPGK (CH2-CH3/Fc v1; SEQ ID NO: 14) | |
| | GGGGSGGGGSGGGGSGGGGSGGGGSGGGGS (G4S linker; SEQ ID NO: 11) | |
| | DCDIEGKDGKQYESVLMVSIDQLLDSMKEIGSNCLNNEFNFFKRHICDANKEGMFLFRAARK | |
| | LRQFLKMNSTGDFDLHLLKVSEGTTILLNCTGQVKGRKPAALGEAQPTKSLEENKSLKEQKK | |
| | LNDLCFLKRLLQEIKTCINKILMGTKEH (IL-7 W142I; SEQ ID NO: 8) | |
| LI17^{142V} (anti- | EVQLVESGGGLVQPGGSLRLSCAASGFTFSSYDMSWVRQAPGKSLEWVATISDAGGYIYYRD | 23 |
| PD-L1 v1) | SVKGRFTISRDNAKNSLYLQMNSLRDEDTAVYICARELPWRYALDYWGQGTTVTVSS (anti- | |
| Format 1 | PD-L1 v1 VH fragment; SEQ ID NO: 1) | |
| Heavy chain | ASTKGPSVFPLAPSSKSTSGGTAALGCLVKDYFPEPVTVSWNSGALTSGVHTFPAVLQSSGL | |
| | YSLSSVVTVPSSSLGTQTYI CNVNHKPSNTKVDKKV (CH1; SEQ ID NO: 13) | |
| | EPKSCDKTHTCPPCP (Hinge v2; SEQ ID NO: 18) | |
| | APELLGGPSVFLFPPKPKDTLMISRTPEVTCVVVDVSHEDPEVKFNWYVDGVEVHNAKTKPR | |
| | EEQYNSTYRVVSVLTVLHQDWLNGKEYKCKVSNKALPAPIEKTISKAKGQPREPQVYTLPPS | |
| | REEMTKNQVSLTCLVKGFYPSDIAVEWESNGQPENNYKTTPPVLDSDGSFFLYSKLTVDKSR | |
| | WQQGNVFSCSVMHEALHNHYTQKSLSLSPGK (CH2-CH3/Fc v1; SEQ ID NO: 14) | |
| | GGGGSGGGGSGGGGSGGGGSGGGGSGGGGS (G4S linker; SEQ ID NO: 11) | |
| | DCDIEGKDGKQYESVLMVSIDQLLDSMKEIGSNCLNNEFNFFKRHICDANKEGMFLFRAARK | |
| | LRQFLKMNSTGDFDLHLLKVSEGTTILLNCTGQVKGRKPAALGEAQPTKSLEENKSLKEQKK | |
| | LNDLCFLKRLLQEIKTCVNKILMGTKEH (IL-7 W142V; SEQ ID NO: 9) | |
| L1I7^{142A} (anti- | EVQLVESGGGLVQPGGSLRLSCAASGFTFSSYDMSWVRQAPGKSLEWVATISDAGGYIYYRD | 24 |
| PD-L1 v1) | SVKGRFTISRDNAKNSLYLQMNSLRDEDTAVYICARELPWRYALDYWGQGTTVTVSS (anti- | |
| Format 1 | PD-L1 v1 VH fragment; SEQ ID NO: 1) | |
| Heavy chain | ASTKGPSVFPLAPSSKSTSGGTAALGCLVKDYFPEPVTVSWNSGALTSGVHTFPAVLQSSGL | |
| | YSLSSVVTVPSSSLGTQTYICNVNHKPSNTKVDKKV (CH1; SEQ ID NO: 13) | |
| | EPKSCDKTHTCPPCP (Hinge v2; SEQ ID NO: 18) | |
| | APELLGGPSVFLFPPKPKDTLMISRTPEVTCVVVDVSHEDPEVKFNWYVDGVEVHNAKTKPR | |
| | EEQYNSTYRVVSVLTVLHQDWLNGKEYKCKVSNKALPAPIEKTISKAKGQPREPQVYTLPPS | |
| | REEMTKNQVSLTCLVKGFYPSDIAVEWESNGQPENNYKTTPPVLDSDGSFFLYSKLTVDKSR | |
| | WQQGNVFSCSVMHEALHNHYTQKSLSLSPGK (CH2-CH3/Fc v1; SEQ ID NO: 14) | |
| | GGGGSGGGGSGGGGSGGGGSGGGGSGGGGS (G4S linker; SEQ ID NO: 11) | |
| | DCDIEGKDGKQYESVLMVSIDQLLDSMKEIGSNCLNNEFNFFKRHICDANKEGMFLFRAARK | |
| | LRQFLKMNSTGDFDLHLLKVSEGTTILLNCTGQVKGRKPAALGEAQPTKSLEENKSLKEQKK | |
| | LNDLCFLKRLLQEIKTCANKILMGTKEH (IL-7 W142A; SEQ ID NO: 10) | |
| L1I7^{WT} (anti-PD- | DIQMTQSPSSLSASVGDRVTITCKASQDVTPAVAWYQQKPGKAPKLLIYSTSSRYTGVPSRF | 25 |
| L1 v1) Format 1 | SGSGSGTDFTFTISSLQPEDIATYYCQQHYTTPLTFGQGTKLEIK (anti-PD-L1 | |
| Light chain | v1/v2/v3 VL; SEQ ID NO: 2) | |
| | RTVAAPSVFIFPPSDEQLKSGTASVVCLLNNFYPREAKVQWKVDNALQGNSQESVTEQDSK | |
| | DSTYSLSSTLTLSKADYEKHKVYACEVTHQGLSSPVTKSFNRGEC (CL; SEQ ID NO: 12) | |

TABLE 1A
Format 1 molecules - SEQ ID NO: for each chain

| | HC | LC |
|---|---|---|
| L1I7^{WT} (anti-PD-L1 v1) | 21 | 25 |
| L1I7^{142I} (anti-PD-L1 v1) | 22 | 25 |
| L1I7^{142V} (anti-PD-L1 v1) | 23 | 25 |
| L1I7^{142A} (anti-PD-L1 v1) | 24 | 25 |

### Example 2: New Format Screening of L1I7 to Improve In Vivo Stability

Two additional formats (Format 2 and Format 3, FIG. 5, Table 2) were designed and screened by a rapid PK study in humanized mouse in order to evaluate the in vivo stability of the L1I7 fusion molecule. The original format, Format 1 (Table 2) which showed in vivo breakdown in cynomolgus was used as a positive control. Briefly, CD34⁺ hematopoietic stem cell (HSC)-transplanted naïve humanized mice were used to mimic the human immune systems in vivo. Three L1I7^{WT} molecules using different formats were injected into the mice intraperitoneally at day 1 with four mice at each group. Serum were collected at predose, 1 hour, day 3 and day 7 after dosing. The concentrations of the whole molecule and the anti-PDL1 part of these L1I7^{WT} formats in serum were measured by ELISA using the similar PK detection methods in cynomolgus.

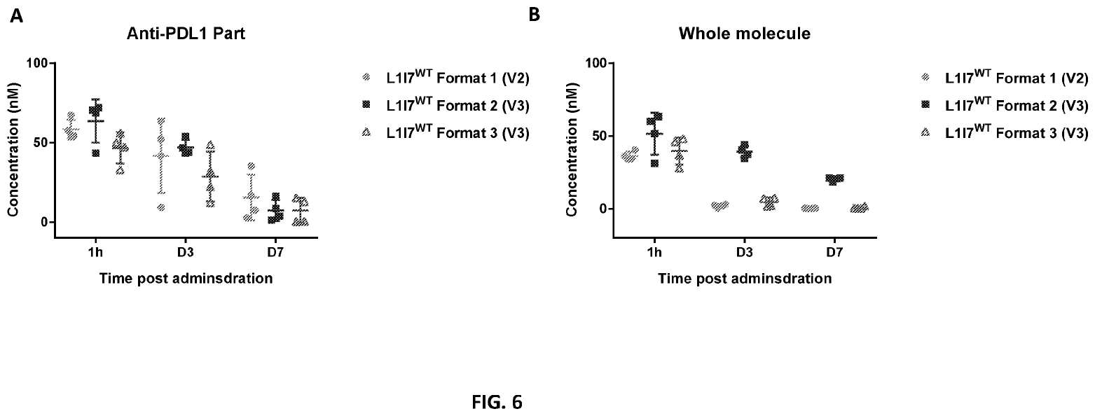

The result showed that the serum concentrations of the anti-PDL1 part of three L1I7^{WT} molecules with different formats were comparable among these groups at each time point (FIG. 6). However, the serum concentrations of the whole molecules of Format 1 and Format 3 were slightly lower than that of Format 2 at 1 hour after dosing and then decreased significantly at day 3 and day 7 compared with that of Format 2 (FIG. 6). These data collectively indicated that the L1I7^{WT} Format 2 was more stable than Format 1 and Format 3 in humanized mouse system.

TABLE 2
L1I7^{WT} (anti-PD-L1 v2/3) Format 1, Format 2 and Format 3 sequences (CDR
residues underlined; mutation sites bolded)

| | | SEQ |
|---|---|---|
| Name | Sequence | ID NO: |
| L1I7^{WT} | EVQLVESGGGLVQPGGSLRLSCAASGFTFSSYDMSWVRQAPGKSLEWVATISDAGGYIYYRD | 26 |
| (anti-PD- | SVKGRFTISRDNAKNSLYLQMNSLRDEDTAVYICAREFGKRYALDYWGQGTTVTVSS (anti- | |
| L1 v2) | PD-L1 v2, VH; SEQ ID NO: 3) | |
| Format 1 | ASTKGPSVFPLAPSSKSTSGGTAALGCLVKDYFPEPVTVSWNSGALTSGVHTFPAVLQSSGL | |
| Heavy | YSLSSVVTVPSSSLGTQTYICNVNHKPSNTKVDKKV (CH1; SEQ ID NO: 13) | |
| chain | EPKSCDKTHTCPPCP (Hinge v2; SEQ ID NO: 18) | |
| | APELLGGPSVFLFPPKPKDTLMISRTPEVTCVVVDVSHEDPEVKFNWYVDGVEVHNAKTKPR | |
| | EEQYNSTYRVVSVLTVLHQDWLNGKEYKCKVSNKALPAPIEKTISKAKGQPREPQVYTLPPS | |
| | REEMTKNQVSLTCLVKGFYPSDIAVEWESNGQPENNYKTTPPVLDSDGSFFLYSKLTVDKSR | |
| | WQQGNVFSCSVMHEALHNHYTQKSLSLSPGK (CH2-CH3/Fc v1; SEQ ID NO: 14) | |
| | GGGGSGGGGSGGGGSGGGGSGGGGSGGGGS (G4S linker; SEQ ID NO: 11) | |
| | DCDIEGKDGKQYESVLMVSIDQLLDSMKEIGSNCLNNEFNFFKRHICDANKEGMFLFRAARK | |
| | LRQFLKMNSTGDFDLHLLKVSEGTTILLNCTGQVKGRKPAALGEAQPTKSLEENKSLKEQKK | |
| | LNDLCFLKRLLQEIKTCWNKILMGTKEH (IL7 wild-type; SEQ ID NO: 7) | |
| L1I7^{WT} | EVQLVESGGGLVQPGGSLRLSCAASGFTFSSYDMSWVRQAPGKSLEWVATISDAGGYIYYSD | 27 |
| (anti-PD- | SVKGRFTISRDNAKNSLYLQMNSLRDEDTAVYICAREFGKRYALDYWGQGTTVTVSS (anti- | |
| L1 v3) | PD-L1 v3, VH; SEQ ID NO: 4) | |
| Format 2 | ASTKGPSVFPLAPSSKSTSGGTAALGCLVKDYFPEPVTVSWNSGALTSGVHTFPAVLQSSGL | |
| Heavy | YSLSSVVTVPSSSLGTQTYICNVNHKPSNTKVDKKV (CH1; SEQ ID NO: 13) | |
| chain-1 | EPKSC (Hinge v1; SEQ ID NO: 17) | |
| (Fab-Fab- | EVQLVESGGGLVQPGGSLRLSCAASGFTFSSYDMSWVRQAPGKSLEWVATISDAGGYIYYSD | |
| Fc) | SVKGRFTISRDNAKNSLYLQMNSLRDEDTAVYICAREFGKRYALDYWGQGTTVTVSS (anti- | |
| | PD-L1 v3, VH; SEQ ID NO: 4) | |
| | ASTKGPSVFPLAPSSKSTSGGTAALGCLVKDYFPEPVTVSWNSGALTSGVHTFPAVLQSSGL | |
| | YSLSSVVTVPSSSLGTQTYICNVNHKPSNTKVDKKV (CH1; SEQ ID NO: 13) | |
| | EPKSCDKTHTCPPCP (Hinge v2; SEQ ID NO: 18) | |
| | APELLGGPSVFLFPPKPKDTLMISRTPEVTCVVVDVSHEDPEVKFNWYVDGVEVHNAKTKPR | |
| | EEQYNSTYRVVSVLTVLHQDWLNGKEYKCKVSNKALPAPIEKTISKAKGQPREPQVYTLPPS | |
| | REEMTKNQVSLWCLVKGFYPSDIAVEWESNGQPENNYKTTPPVLDSDGSFFLYSKLTVDKSR | |
| | WQQGNVFSCSVMHEALHNHYTQKSLSLSPGK (CH2-CH3 v2; SEQ ID NO: 15) | |
| L1I7^{WT} | EVQLVESGGGLVQPGGSLRLSCAASGFTFSSYDMSWVRQAPGKSLEWVATISDAGGYIYYRD | 37 |
| (anti-PD- | SVKGRFTISRDNAKNSLYLQMNSLRDEDTAVYICARELPWRYALDYWGQGTTVTVSS (anti- | |
| L1 v1) | PD-L1 v1, VH; SEQ ID NO: 1) | |
| Format 2 | ASTKGPSVFPLAPSSKSTSGGTAALGCLVKDYFPEPVTVSWNSGALTSGVHTFPAVLQSSGL | |
| Heavy | YSLSSVVTVPSSSLGTQTYICNVNHKPSNTKVDKKV (CH1; SEQ ID NO: 13) | |
| chain-1 | EPKSC (Hinge v1; SEQ ID NO: 17) | |
| (Fab-Fab- | EVQLVESGGGLVQPGGSLRLSCAASGFTFSSYDMSWVRQAPGKSLEWVATISDAGGYIYYSD | |
| Fc) | SVKGRFTISRDNAKNSLYLQMNSLRDEDTAVYICAREFGKRYALDYWGQGTTVTVSS (anti- | |
| | PD-L1 v3, VH; SEQ ID NO: 4) | |
| | ASTKGPSVFPLAPSSKSTSGGTAALGCLVKDYFPEPVTVSWNSGALTSGVHTFPAVLQSSGL | |
| | YSLSSVVTVPSSSLGTQTYICNVNHKPSNTKVDKKV (CH1; SEQ ID NO: 13) | |
| | EPKSCDKTHTCPPCP (Hinge v2; SEQ ID NO: 18) | |
| | APELLGGPSVFLFPPKPKDTLMISRTPEVTCVVVDVSHEDPEVKFNWYVDGVEVHNAKTKPR | |
| | EEQYNSTYRVVSVLTVLHQDWLNGKEYKCKVSNKALPAPIEKTISKAKGQPREPQVYTLPPS | |
| | REEMTKNQVSLWCLVKGFYPSDIAVEWESNGQPENNYKTTPPVLDSDGSFFLYSKLTVDKSR | |
| | WQQGNVFSCSVMHEALHNHYTQKSLSLSPGK (CH2-CH3 v2; SEQ ID NO: 15) | |
| L1I7^{WT} | DCDIEGKDGKQYESVLMVSIDQLLDSMKEIGSNCLNNEFNFFKRHICDANKEGMFLFRAARK | 28 |
| Format 2 | LRQFLKMNSTGDFDLHLLKVSEGTTILLNCTGQVKGRKPAALGEAQPTKSLEENKSLKEQKK | |
| Heavy | LNDLCFLKRLLQEIKTCWNKILMGTKEH (IL7 wild-type; SEQ ID NO: 7) | |
| chain-2 | EPKSADKTHTCPPCP (Hinge v3; SEQ ID NO: 19) | |
| (IL7-Fc) | APELLGGPSVFLFPPKPKDTLMISRTPEVTCVVVDVSHEDPEVKFNWYVDGVEVHNAKTKPR | |
| | EEQYNSTYRVVSVLTVLHQDWLNGKEYKCKVSNKALPAPIEKTISKAKGQPREPQVYTLPPS | |
| | REEMTKNQVSLSCAVKGFYPSDIAVEWESNGQPENNYKTTPPVLDSDGSFFLVSKLTVDKSR | |
| | WQQGNVFSCSVMHEALHNHYTQKSLSLSPGK (CH2-CH3 v3; SEQ ID NO: 16) | |
| L1I7^{WT} | DCDIEGKDGKQYESVLMVSIDQLLDSMKEIGSNCLNNEFNFFKRHICDANKEGMFLFRAARK | 29 |
| (anti-PD- | LRQFLKMNSTGDFDLHLLKVSEGTTILLNCTGQVKGRKPAALGEAQPTKSLEENKSLKEQKK | |
| L1 v3) | LNDLCFLKRLLQEIKTCWNKILMGTKEH (IL7 wild-type; SEQ ID NO: 7) | |
| Format 3 | GGGGSGGGGSGGGGSGGGGSGGGGSGGGGS (G4S linker; SEQ ID NO: 11) | |
| Heavy | EVQLVESGGGLVQPGGSLRLSCAASGFTFSSYDMSWVRQAPGKSLEWVATISDAGGYIYYSD | |
| chain | SVKGRFTISRDNAKNSLYLQMNSLRDEDTAVYICAREFGKRYALDYWGQGTTVTVSS (anti- | |
| | PD-L1 v3, VH; SEQ ID NO: 4) | |
| | ASTKGPSVFPLAPSSKSTSGGTAALGCLVKDYFPEPVTVSWNSGALTSGVHTFPAVLQSSGL | |
| | YSLSSVVTVPSSSLGTQTYICNVNHKPSNTKVDKKV (CH1; SEQ ID NO: 13) | |
| | EPKSCDKTHTCPPCP (Hinge v2; SEQ ID NO: 18) | |
| | APELLGGPSVFLFPPKPKDTLMISRTPEVTCVVVDVSHEDPEVKFNWYVDGVEVHNAKTKPR | |
| | EEQYNSTYRVVSVLTVLHQDWLNGKEYKCKVSNKALPAPIEKTISKAKGQPREPQVYTLPPS | |
| | REEMTKNQVSLTCLVKGFYPSDIAVEWESNGQPENNYKTTPPVLDSDGSFFLYSKLTVDKSR | |
| | WQQGNVFSCSVMHEALHNHYTQKSLSLSPGK (CH2-CH3/Fc v1; SEQ ID NO: 14) | |

TABLE 2A
Anti-PD-L1 v3 in different formats - SEQ ID NO: for each chain

| | HC1 | HC2 | LC |
|---|---|---|---|
| L1I7^{WT}(anti-PD-L1 v2) Format 1 | 26 | | 25 |
| L1I7^{WT}(anti-PD-L1 v3) Format 2 | 27 | 28 | 25 |
| L1I7^{WT}(anti-PD-L1 v3) Format 3 | 29 | | 25 |

TABLE 3
L1I7^{WT} (anti-PD-L1 v4) Format 1, Format 2 and Format 3 sequences (CDR
residues underlined; mutation sites bolded)

| | | SEQ ID |
|---|---|---|
| Name | Sequence | NO: |
| L1I7^{WT} | QVQLLESGGGLVQPGGSLRLSCAASGFTFSSYWMSWVRQAPGKGLEWVANIKQDGSEKYYVD | 30 |
| (anti-PD- | SVKGRFTISRDNAKNSLYLQMNSLRAEDTAVYYCARVALWDDAFDIWGQGTMVTVSS (anti- | |
| L1 v4) | PD-L1 v4 VH; SEQ ID NO: 5) | |
| Format 1 | ASTKGPSVFPLAPSSKSTSGGTAALGCLVKDYFPEPVTVSWNSGALTSGVHTFPAVLQSSGL | |
| Heavy | YSLSSVVTVPSSSLGTQTYICNVNHKPSNTKVDKKV (CH1; SEQ ID NO: 13) | |
| chain | EPKSCDKTHTCPPCP (Hinge v2; SEQ ID NO: 18) | |
| | APELLGGPSVFLFPPKPKDTLMISRTPEVTCVVVDVSHEDPEVKFNWYVDGVEVHNAKTKPR | |
| | EEQYNSTYRVVSVLTVLHQDWLNGKEYKCKVSNKALPAPIEKTISKAKGQPREPQVYTLPPS | |
| | REEMTKNQVSLTCLVKGFYPSDIAVEWESNGQPENNYKTTPPVLDSDGSFFLYSKLTVDKSR | |
| | WQQGNVFSCSVMHEALHNHYTQKSLSLSPGK (CH2-CH3/Fc v1; SEQ ID NO: 14) | |
| | GGGGSGGGGSGGGGSGGGGSGGGGSGGGGS (G4S linker; SEQ ID NO: 11) | |
| | DCDIEGKDGKQYESVLMVSIDQLLDSMKEIGSNCLNNEFNFFKRHICDANKEGMFLFRAARK | |
| | LRQFLKMNSTGDFDLHLLKVSEGTTILLNCTGQVKGRKPAALGEAQPTKSLEENKSLKEQKK | |
| | LNDLCFLKRLLQEIKTCWNKILMGTKEH (IL7 wild-type; SEQ ID NO: 7) | |
| L1I7^{WT} | QVQLLESGGGLVQPGGSLRLSCAASGFTFSSYWMSWVRQAPGKGLEWVANIKQDGSEKYYVD | 31 |
| (anti-PD- | SVKGRFTISRDNAKNSLYLQMNSLRAEDTAVYYCARVALWDDAFDIWGQGTMVTVSS (anti- | |
| L1 v4) | PD-L1 v4 VH; SEQ ID NO: 5) | |
| Format 2 | ASTKGPSVFPLAPSSKSTSGGTAALGCLVKDYFPEPVTVSWNSGALTSGVHTFPAVLQSSGL | |
| Heavy | YSLSSVVTVPSSSLGTQTYICNVNHKPSNTKVDKKV (CH1; SEQ ID NO: 13) | |
| chain-1 | EPKSC (Hinge v1; SEQ ID NO: 17) | |
| (Fab-Fab- | QVQLLESGGGLVQPGGSLRLSCAASGFTFSSYWMSWVRQAPGKGLEWVANIKQDGSEKYYVD | |
| Fc) | SVKGRFTISRDNAKNSLYLQMNSLRAEDTAVYYCARVALWDDAFDIWGQGTMVTVSS (anti- | |
| | PD-L1 v4 VH; SEQ ID NO: 5) | |
| | ASTKGPSVFPLAPSSKSTSGGTAALGCLVKDYFPEPVTVSWNSGALTSGVHTFPAVLQSSGL | |
| | YSLSSVVTVPSSSLGTQTYICNVNHKPSNTKVDKKV (CH1; SEQ ID NO: 13) | |
| | EPKSCDKTHTCPPCP (Hinge v2; SEQ ID NO: 18) | |
| | APELLGGPSVFLFPPKPKDTLMISRTPEVTCVVVDVSHEDPEVKFNWYVDGVEVHNAKTKPR | |
| | EEQYNSTYRVVSVLTVLHQDWLNGKEYKCKVSNKALPAPIEKTISKAKGQPREPQVYTLPPS | |
| | REEMTKNQVSLWCLVKGFYPSDIAVEWESNGQPENNYKTTPPVLDSDGSFFLYSKLTVDKSR | |
| | WQQGNVFSCSVMHEALHNHYTQKSLSLSPGK (CH2-CH3 v2; SEQ ID NO: 15) | |
| L1I7^{WT} | DCDIEGKDGKQYESVLMVSIDQLLDSMKEIGSNCLNNEFNFFKRHICDANKEGMFLFRAARK | 32 |
| (anti-PD- | LRQFLKMNSTGDFDLHLLKVSEGTTILLNCTGQVKGRKPAALGEAQPTKSLEENKSLKEQKK | |
| L1 v4) | LNDLCFLKRLLQEIKTCWNKILMGTKEH (IL7 wild-type; SEQ ID NO: 7) | |
| Format 3 | GGGGSGGGGSGGGGSGGGGSGGGGSGGGGS (G4S linker; SEQ ID NO: 11) | |
| Heavy | QVQLLESGGGLVQGGSLRLSCAASGFTFSSYWMSWVRQAPGKGLEWVANIKQDGSEKYYVD | |
| chain | SVKGRFTISRDNAKNSLYLQMNSLRAEDTAVYYCARVALWDDAFDIWGQGTMVTVSS (anti- | |
| | PD-L1 v4 VH; SEQ ID NO: 5) | |
| | ASTKGPSVFPLAPSSKSTSGGTAALGCLVKDYFPEPVTVSWNSGALTSGVHTFPAVLQSSGL | |
| | YSLSSVVTVPSSSLGTQTYICNVNHKPSNTKVDKKV (CH1; SEQ ID NO: 13) | |
| | EPKSCDKTHTCPPCP (Hinge v2; SEQ ID NO: 18) | |
| | APELLGGPSVFLFPPKPKDTLMISRTPEVTCVVVDVSHEDPEVKFNWYVDGVEVHNAKTKPR | |
| | EEQYNSTYRVVSVLTVLHQDWLNGKEYKCKVSNKALPAPIEKTISKAKGQPREPQVYTLPPS | |
| | REEMTKNQVSLTCLVKGFYPSDIAVEWESNGQPENNYKTTPPVLDSDGSFFLYSKLTVDKSR | |
| | WQQGNVFSCSVMHEALHNHYTQKSLSLSPGK (CH2-CH3/Fc v1; SEQ ID NO: 14) | |
| L1I7^{WT} | DIQMTQSPSTLSASVGDRVIITCRASRGISSWLAWYQQKPGKAPNLLISKASSLESGVPSRF | 33 |
| (anti-PD- | SGSGSGTDFTLTISSLQPEDFATYYCQQSSSIPLTFGGGTKVEIK (anti-PD-L1 v4 VL; | |
| L1 v4) | SEQ ID NO: 6) | |
| Format | RTVAAPSVFIFPPSDEQLKSGTASVVCLLNNFYPREAKVQWKVDNALQSGNSQESVTEQDSK | |
| 1/Format | DSTYSLSSTLTLSKADYEKHKVYACEVTHQGLSSPVTKSFNRGEC (CL; SEQ ID NO: 12) | |
| 2/Format 3 | | |
| Light chain | | |

TABLE 3A
Anti-PD-L1 v4 in different formats - SEQ ID NO: for each chain

| | HC1 | HC2 | LC |
|---|---|---|---|
| L1I7^{WT}(anti-PD-L1 v4) Format 1 | 30 | | 33 |
| L1I7^{WT}(anti-PD-L1 v4) Format 2 | 31 | 28 | 33 |
| L1I7^{WT}(anti-PD-L1 v4) Format 3 | 32 | | 33 |

### Example 3: In Vivo PK/PD Study of Format 2 Anti-PDL1-IL7 in Cynomolgus Monkey

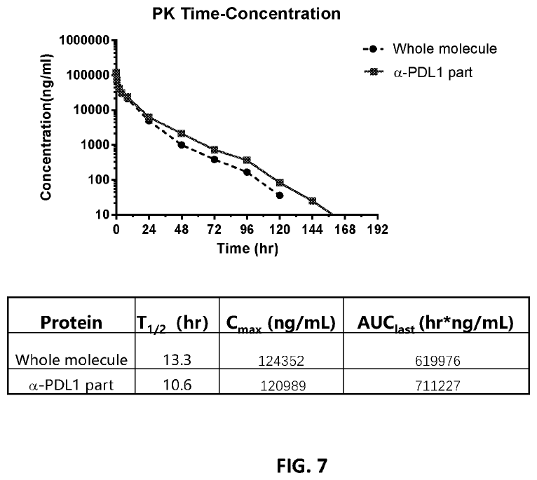

To validate the PK/PD profile of L1I7^{WT} Format 2 in cynomolgus monkey, naïve cynomolgus monkeys (n=2, 1 male/1 female) were intravenously injected with L1I7^{WT} Format 2 (Table 2) at 15 mg/kg for single dose. The concentrations of the whole molecule and the anti-PDL1 part of L1I7^{WT} in serum were measured by ELISA as described above. The concentration-time curves in serum and the pharmacokinetic parameters of L1I7^{WT} Format 2 after a single dose at 15 mg/kg in cynomolgus monkeys were shown in FIG. 7. The results showed that curves of the whole molecule and the anti-PDL1 part of L1I7^{WT} Format 2 paralleled with each other. The T_{1/2} of the whole molecule and the anti-PDL1 part was also similar with each other (T_{1/2} whole vs. anti-PDL1 of L1I7^{WT} Format 2: 13.3 vs. 10.6 hours), indicating stability of this new format in cynomolgus. In addition, the T_{1/2} of the whole molecule of the L1I7^{WT} Format 2 was prolonged compared with that of the previous L1I7^{WT} Format 1 (T_{1/2} whole molecule of L1I7^{WT} Format 2 vs. Format 1: 7.1 vs. 13.3 hours), indicating the contribution on the improved stability by the new format.

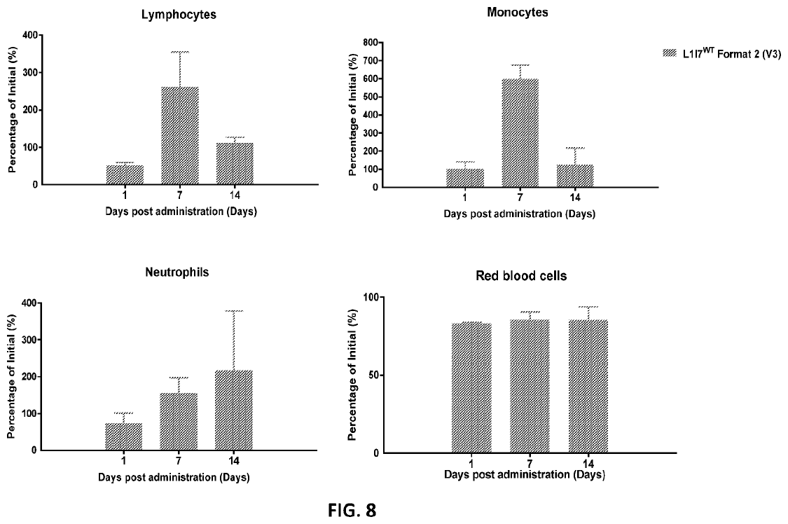

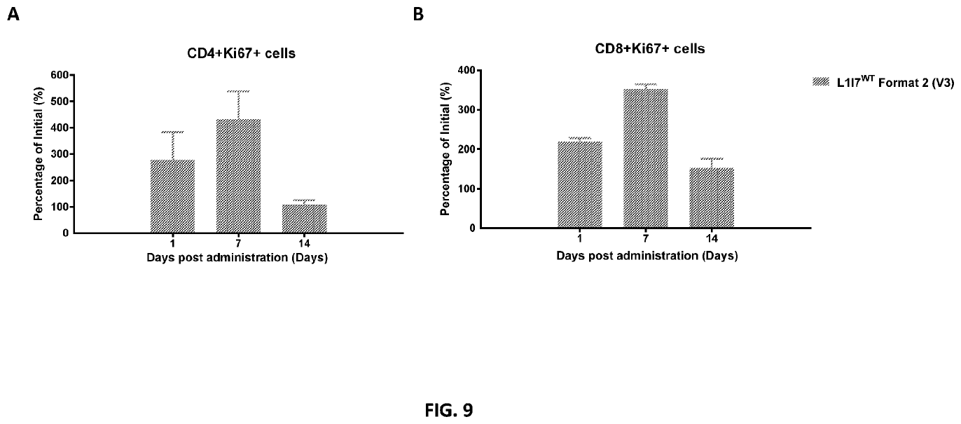

For pharmacodynamics analysis, the absolute cell counts of individual immune cell types at different time points were analyzed as above. As shown in FIG. 8, the lymphocyte cell counts significantly increased up to about 2.5 folds at day 7 after a single dose of L1I7^{WT} Format 2. The role of L1I7^{WT} Format 2 treatment on promotion of lymphocyte cell counts was much stronger than that of L1I7^{WT} Format 1 treatment, which showed no obvious change after 7 days after the first dose and only about 1.5 folds increase after the second dose (FIG. 2). A significant increase of monocytes cell counts was also observed 7 days after dosing although it was not expected. On the contrary, a slight increase of neutrophil cell counts and no change in the amounts of red blood cells were observed (FIG. 8). The percentages of Ki67⁺CD4⁺ and Ki67⁺CD8⁺ T cells were largely promoted by L1I7^{WT} Format 2 at day 1 and day 7 after dosing (FIG. 9), which also showed more intensive changes than that induced by L1I7^{WT} Format 1 (FIG. 3). These data indicated that L1I7^{WT} Format 2 was stable in vivo and therefore induced more significant downstream effect compared with the previous unstable L1I7^{WT} Format 1.

### Example 4: PDL1 Binding Activity of Anti-PDL1-IL7 Fusion Molecules

To evaluate the in vitro properties of L1I7 Format 2 wild type and mutant molecules, the fusion proteins were constructed using two different anti-PDL1 antibody sequences and the corresponding IL7 WT or mutant part (Table 2-4).

TABLE 4
Sequences of L1I7 Format 2 mutant molecules (mutation sites bolded)

| | | SEQ |
|---|---|---|
| Name | Sequence | ID NO: |
| L1I7^{142I} | DCDIEGKDGKQYESVLMVSIDQLLDSMKEIGSNCLNNEFNEFKRHICDANKEGMFLFRAARK | 34 |
| Format 2 | LRQFLKMNSTGDFDLHLLKVSEGTTILLNCTGQVKGRKPAALGEAQPTKSLEENKSLKEQKK | |
| Heavy | LNDLCFLKRLLQEIKTCINKILMGTKEH (IL-7 W142I; SEQ ID NO: 8) | |
| chain-2 | EPKSADKTHTCPPCP (Hinge v3; SEQ ID NO: 19) | |
| (IL7-Fc) | APELLGGPSVFLFPPKPKDTLMISRTPEVTCVVVDVSHEDPEVKFNWYVDGVEVHNAKTKPR | |
| | EEQYNSTYRVVSVLTVLHQDWLNGKEYKCKVSNKALPAPIEKTISKAKGQPREPQVYTLPPS | |
| | REEMTKNQVSLSCAVKGFYPSDIAVEWESNGQPENNYKTTPPVLDSDGSFFLVSKLTVDKSR | |
| | WQQGNVFSCSVMHEALHNHYTQKSLSLSPGK (CH2-CH3 v3; SEQ ID NO: 16) | |
| L1I7^{142V} | DCDIEGKDGKQYESVLMVSIDQLLDSMKEIGSNCLNNEFNEFKRHICDANKEGMFLFRAARK | 35 |
| Format 2 | LRQFLKMNSTGDFDLHLLKVSEGTTILLNCTGQVKGRKPAALGEAQPTKSLEENKSLKEQKK | |
| Heavy | LNDLCFLKRLLQEIKTCVNKILMGTKEH (IL-7 W142V; SEQ ID NO: 9) | |
| chain-2 | EPKSADKTHTCPPCP (Hinge v3; SEQ ID NO: 19) | |
| (IL7-Fc) | APELLGGPSVFLFPPKPKDTLMISRTPEVTCVVVDVSHEDPEVKFNWYVDGVEVHNAKTKPR | |
| | EEQYNSTYRVVSVLTVLHQDWLNGKEYKCKVSNKALPAPIEKTISKAKGQPREPQVYTLPPS | |
| | REEMTKNQVSLSCAVKGFYPSDIAVEWESNGQPENNYKTTPPVLDSDGSFFLVSKLTVDKSR | |
| | WQQGNVFSCSVMHEALHNHYTQKSLSLSPGK (CH2-CH3 v3; SEQ ID NO: 16) | |
| L1I7^{142A} | DCDIEGKDGKQYESVLMVSIDQLLDSMKEIGSNCLNNEFNEFKRHICDANKEGMFLFRAARK | 36 |
| Format 2 | LRQFLKMNSTGDFDLHLLKVSEGTTILLNCTGQVKGRKPAALGEAQPTKSLEENKSLKEQKK | |
| Heavy | LNDLCFLKRLLQEIKTCANKILMGTKEH (IL-7 W142A; SEQ ID NO: 10) | |
| chain-2 | EPKSADKTHTCPPCP (Hinge v3; SEQ ID NO: 19) | |
| (IL7-Fc) | APELLGGPSVFLFPPKPKDTLMISRTPEVTCVVVDVSHEDPEVKFNWYVDGVEVHNAKTKPR | |
| | EEQYNSTYRVVSVLTVLHQDWLNGKEYKCKVSNKALPAPIEKTISKAKGQPREPQVYTLPPS | |
| | REEMTKNQVSLSCAVKGFYPSDIAVEWESNGQPENNYKTTPPVLDSDGSFFLVSKLTVDKSR | |
| | WQQGNVFSCSVMHEALHNHYTQKSLSLSPGK (CH2-CH3 v3; SEQ ID NO: 16) | |

TABLE 4A
Format 2 molecules - SEQ ID NO: for each chain

| | HC1 | HC2 | LC |
|---|---|---|---|
| L1I7^{142I}(anti-PD-L1 v3) Format 2 | 27 | 34 | 25 |
| L1I7^{142V} (anti-PD-L1 v3) Format 2 | 27 | 35 | 25 |
| L1I7^{142A} (anti-PD-L1 v3) Format 2 | 27 | 36 | 25 |
| LI17^{142I} (anti-PD-L1 v4) Format 2 | 31 | 34 | 33 |
| L1I7^{142V} (anti-PD-L1 v4) Format 2 | 31 | 35 | 33 |
| L1I7^{142A} (anti-PD-L1 v4) Format 2 | 31 | 36 | 33 |

The binding affinity of the L1I7 Format 2 fusion molecule to recombinant human his-tagged PD-L1 protein was tested by BIACORE® using a capture method. The L1I7 Format 2 molecules or anti-PDL1 monoclonal antibodies were captured by protein A chip. A series of dilutions of human PD-L1 protein were injected over captured antibody at a flow rate of 10 μL/min. The antigen was allowed to associate for 120 s and dissociate for 300 s (anti-PDL1 v3) or 600 s (anti-PDL1 v4). All the experiments were carried out on a Biacore T200. Data analysis was carried out using Biacore T200 evaluation software. The data show that the PDL1 binding affinity was not compromised in the L1I7 Format 2 molecules when compared with its corresponding anti-PDL1 antibody, indicating the compatibility of effective binding to PD-L1 by L1I7 fusion molecules (Table 5).

TABLE 5
Affinity of L1I7 Format 2 molecule compared
with anti-PDL1 monoclonal antibody

| | Analyte | | |
|---|---|---|---|
| | hPDL1-his | | |
| Ligand | ka (1/Ms) | kd (1/s) | KD (M) |
| α-PDL1(anti-PD-L1 v3) | 8.069E+4 | 1.641E−4 | 2.033E−9 |
| L1I7^{WT}(anti-PD-L1 v3) | 8.145E+4 | 2.043E−4 | 2.508E−9 |
| α-PDL1(anti-PD-L1 v4) | 8.382 + 5 | 7.949E−4 | 9.483E−10 |
| L1I7^{WT}(anti-PD-L1 v4) | 7.995E+5 | 7.451E−4 | 9.320E−10 |

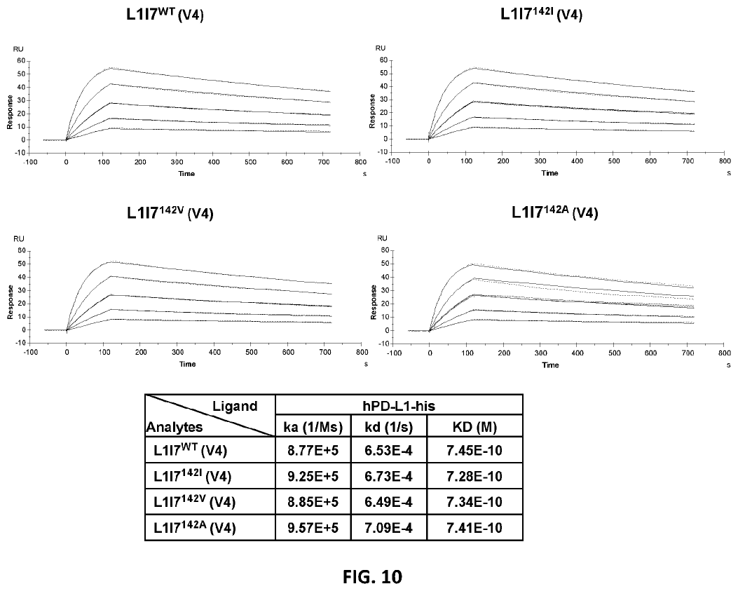

To detect whether the mutations on IL7 molecules affect the PD-L1 binding affinity, this example determined the affinity of the L1I7 Format 2 molecules to his-tagged human PD-L1 by Biacore T200. The data showed that all L1I7 mutant Format 2 molecules retained the similar PD-L1 affinity to L1I7^{WT} molecule as well as the parental anti-PD-L1 (V4) antibody (FIG. 10 and Table 5).

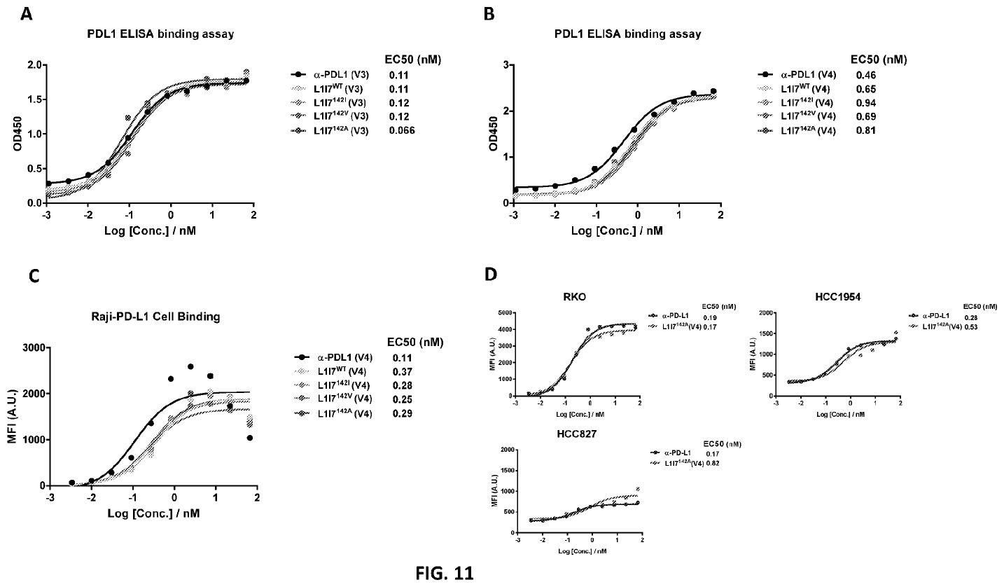

To evaluate the antigen binding property to PD-L1, the L1I7 Format 2 molecules were analyzed for their binding to PDL1 protein by ELISA or cell-surface expressed PD-L1 by flow cytometry. Briefly, for ELISA-based binding, his-tagged PDL1 was used as coating protein. L1I7 fusion proteins or the parental anti-PD-L1 antibody were captured by PDL1 and detected by secondary antibody. The data showed that comparable binding properties of L1I7 Format 2 molecules to their respective anti-PDL1 antibodies (FIGS. 11A and 11B).

For cell-based binding, PDL1-overexpressed Raji cells (Raji-PD-L1) were first incubated with 3-fold serial diluted L1I7 or anti-PD-L1 mAb antibodies starting at 66.7 nM on ice for 30 mins. After washing by FACS staining buffer, the PE-conjugated anti-human IgG Fc specific antibody was added to each well and incubated on ice for 30 mins. The MFI of PE were evaluated by FACS Celesta. As shown in FIG. 11C, all the L1I7 fusion molecules showed relatively comparable binding capability to cell surface PD-L1 to that of their parental PD-L1 antibody.

To further validate the binding property of L1I7 to cell-surface PD-L1 expressed on human tumor cells, we selected several human tumor cell lines including colon carcinoma cell line RKO, breast cancer cell line HCC1954 and lung cancer cell line HCC827 for validation. As shown in FIG. 11D, L1I7^{142A} exhibited comparable binding ability to PD-L1 expressed on human tumor cell to that of its parental PD-L1 antibody. These data collectively indicated that L1I7 Format 2 fusion molecules could binding to PD-L1 protein expressed on target tumor cells as efficiently as the parental anti-PD-L1 mAb.

### Example 5: PDL1 Antagonist Activity of Anti-PDL1-IL7 Fusion Molecules

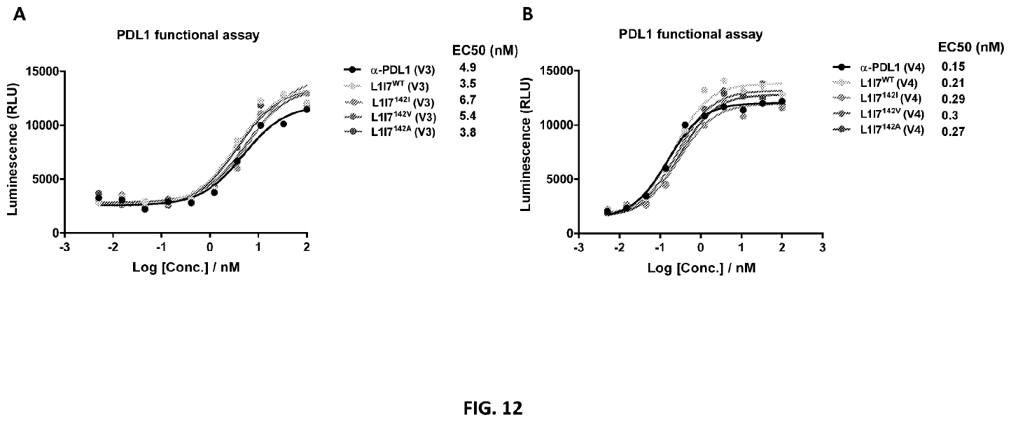

To evaluate the PDL1 antagonist effect of the L1I7 Format 2 fusion molecules, PDL1 cell-based functional assay was performed. Jurkat cells overexpressed PD1 (Jurkat-PD1) was cocultured with Raji cells overexpressed with PDL1 (Raji-PDL1) in the presence of a super antigen Staphylococcal Enterotoxin (SEE). SEE stimulated the IL2 production by Jurkat T cells in the presence of Raji cells through ligation of MHCII on Raji and TCR molecules on Jurkat. PDL1 exogenous expressed on Raji-PDL1 cells bound to PD1 overexpressed by Jurkat T cells and inhibited the IL2 production by Jurkat. The anti-PDL1 monoclonal antibody or L1I7 Format 2 molecules reversed IL2 production suppressed by PD1/PDL1 pathway. As shown in FIG. 12, all L1I7 fusion molecules showed comparable PDL1 antagonist function with their parental anti-PDL1 mAb.

### Example 6: Attenuated IL7 Activity of Anti-PDL1-IL7 Fusion Molecules

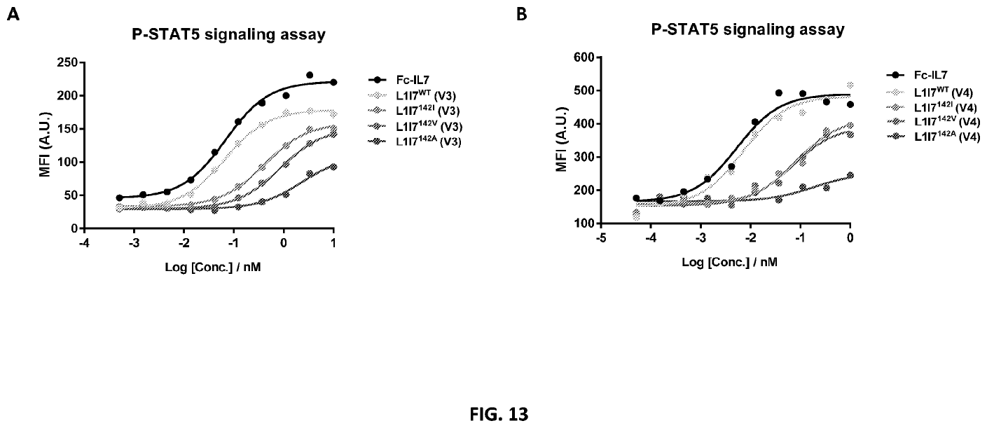

To confirm the attenuation of IL7 activity of the L1I7 Format 2 fusion molecules on primary CD4⁺ T cells, IL-7-IL7 receptor (IL-7R) ligation induced downstream P-STAT5 signaling was examined. Briefly, human PBMCs were treated with L1I7 Format 2 molecules or Fc-IL7 as the positive control at the indicated concentration for 15 mins. P-STAT5 level was detected by FACS staining. As shown in FIG. 13, all these mutant L1I7 fusion molecules showed attenuated P-STAT5 activation compared with L1I7^{WT} and Fc-IL-7 cytokine, with L1I7^{142A} showing the weakest P-STAT5 induction, indicating the reduction of IL7 activity of all the L1I7 mutant molecules.

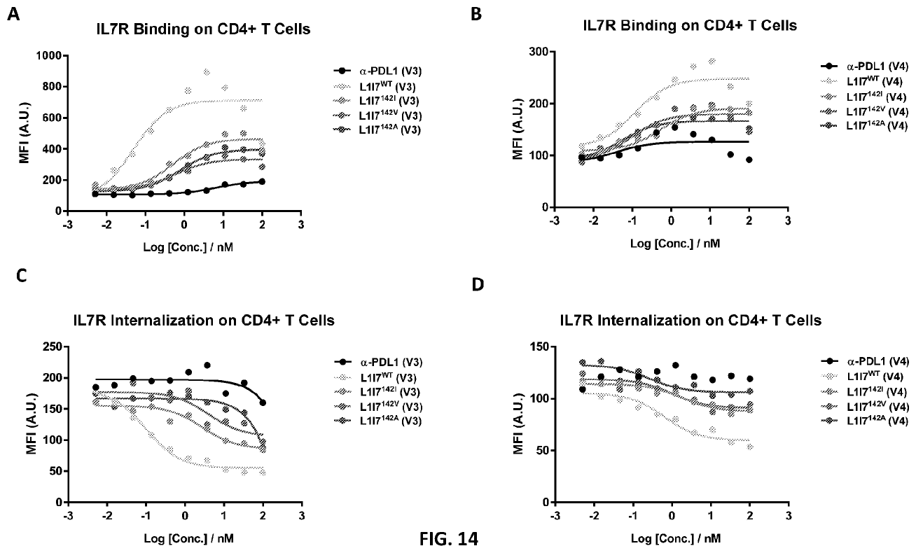

In order to explain the mechanism of attenuated IL7 activities of the serial mutant anti-PDL1-IL7 molecules, IL7R binding and IL-7-IL-7R ligation-mediated internalization were evaluated. Briefly, for IL7R binding assay, human primary CD4⁺ T cells were incubated with various anti-PDL1-IL7 fusion molecules at 4° C. for 30 mins. PE-conjugated anti-human Fc secondary antibody were used to detect the L1I7 fusion molecules that bound to the IL7R of CD4⁺ T cells by FACS. As shown in FIGS. 14A and 14B, all of the three mutant L1I7 fusion molecules with decreased IL7 activities had reduced IL7R binding compared with L1I7^{WT}. For ligation-mediated receptor internalization assay, human primary CD4⁺ T cells were cocultured with fusion molecules at 37° C. for 15 mins to induce internalization. PE-cy7-conjugated-anti-CD127 (IL7Rα) antibody was used to detect the surface IL7Rα by FACS. Similar to the trends of IL7R binding, those fusion molecules with reduced IL7R binding potency had compromised IL7R internalization (FIGS. 14C and 14D), indicating attenuated IL7 signaling transduction.

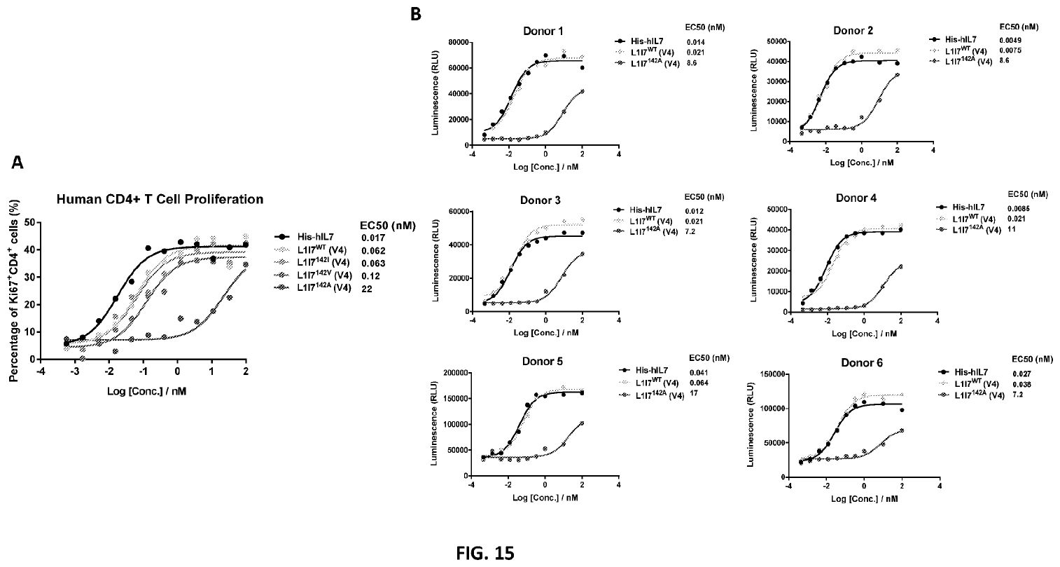

To validate the activity of attenuated L1I7 Format 2 fusion molecules on promoting human primary CD4⁺ T cells proliferation, CD4⁺ T cells were purified from PBMCs derived from healthy donors and treated the cells with L1I7^{WT}, mutant L1I7 molecules or his-tagged human recombinant IL-7 cytokine for 1 week. T cell proliferation was detected by either intracellular staining of cell proliferation marker Ki67 or using a CellTiter-Glo® Luminescent Cell Viability Assay kit (Promega). Consistent with the above findings, this example observed an almost 300 folds decrease in EC50 in promoting CD4⁺ T cell proliferation by L1I7^{142A} compared with L1I7^{WT} (FIG. 15A). Notably, L1I7^{142I} and L1I7^{142V} did not show significantly compromised T cell proliferation compared to L1I7^{WT} and IL-7 despite convincing evidence of impaired IL-7R binding, internalization and downstream signaling.

To further confirm whether different donors showed similar responses to L1I7^{142A}-induced T cell proliferation, this example performed CD4⁺ T cell proliferation assay by using CD4⁺ T cell isolated from PBMCs of six healthy donors. This example observed about 200-1000 folds decrease in EC50 and 30-50% loss in maximum T cell proliferation level in promoting CD4⁺ T cell proliferation by L1I7^{142A} compared with L1I7^{WT} (FIG. 15B). These findings indicated that L1I7^{142A} Format 2 molecule showed IL-7 based functional attenuation despite human heterogenicity. Thus, we have developed a series of L1I7 molecules with IL-7 activity attenuated to varying degrees.

### Example 7: Synergistic Stimulation of Anti-PDL1-IL7 Fusion Molecule on Human T Cell Function

To evaluate the in vitro synergistic function of L1I7 fusion molecules, the response of human T cells was assessed in a mixed lymphocyte reaction setting. Human DCs were differentiated from CD14⁺ monocytes in the presence of GM-CSF and IL-4 for 7 days. CD4⁺ T cells isolated from another donor were then co-cultured with the DCs and serial dilutions of L1I7 fusion molecules or their parental molecules.

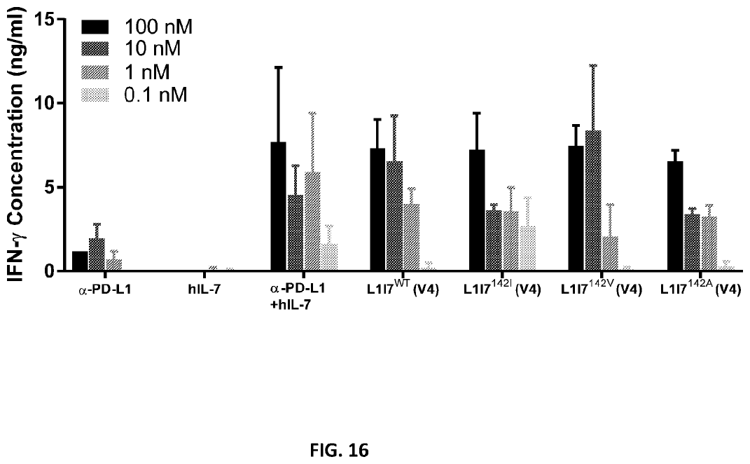

At day 5 post-inoculation, the culture supernatant was assayed for IFNγ production. The results indicated L1I7^{WT} and mutants showed superior efficacy than anti-PDL1 mAb or IL7 on enhancing human T cell function (FIG. 16). L1I7 variants with reduced IL7 potency, showed comparable potency as L1I7 on human T cell response. Therefore, the fusion molecules exhibited synergistic effects on PDL1 antagonism and IL7 activity. Further, the full IL7 activity was not required for the synergistic effects. The anti-PDL1-IL7 molecules with reduced IL7 activity that retained strong synergistic effect on immune-stimulation may actually have better safety profile in the future clinics.

### Example 8: In Vivo Distribution of Anti-PDL1-IL7 Activity

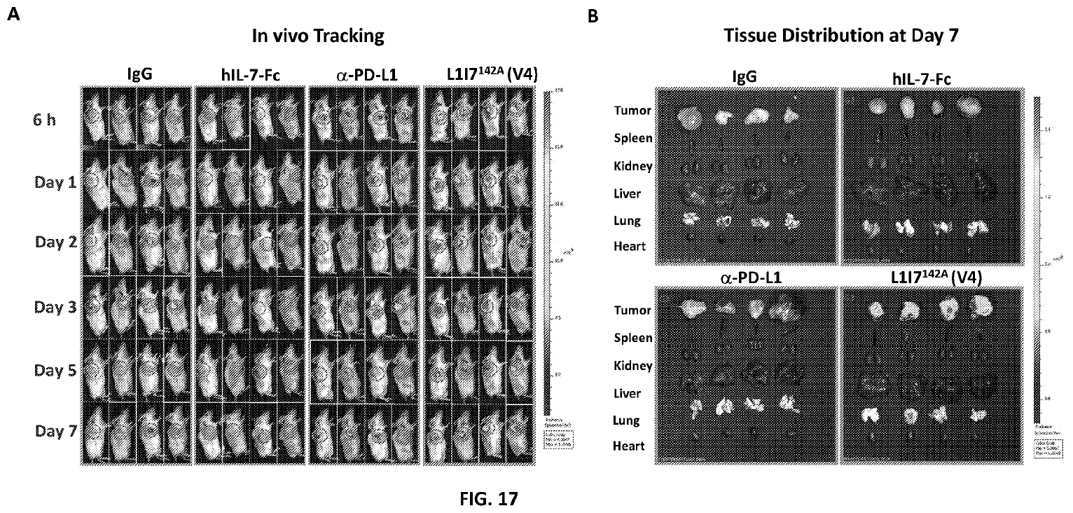

To determine the distribution of anti-PDL1-IL7 fusion molecule in vivo, an in vivo tracking assay was conducted. Briefly, ICG-labeled anti-PDL1 mAb, L1I7^{142A} or human IL7-Fc were injected intravenously into PBMC humanized mice transplanted s.c. with HCC1954 tumor cells when tumor size reached around 700 mm³. Imaging systems were used to capture the fluorescence signal at different time intervals. As shown in FIG. 17A, similar to anti-PDL1 mAb, L1I7^{142A} significantly enriched in the tumor site whereas the hIL7-Fc was widely spread during the period of the observation. Tissue distribution also indicated the preferential location of L1I7 in tumor site in spite of other tissues even at Day 7 after administration (FIG. 17B). These data collectively showed the selective and specific distribution of L1I7 fusion molecule, demonstrating the reduced systemic effect of IL7 in the fusion molecules.

### Example 9: Bi-Specific Binding Property of L1I7 to PD-L1 and IL-7R

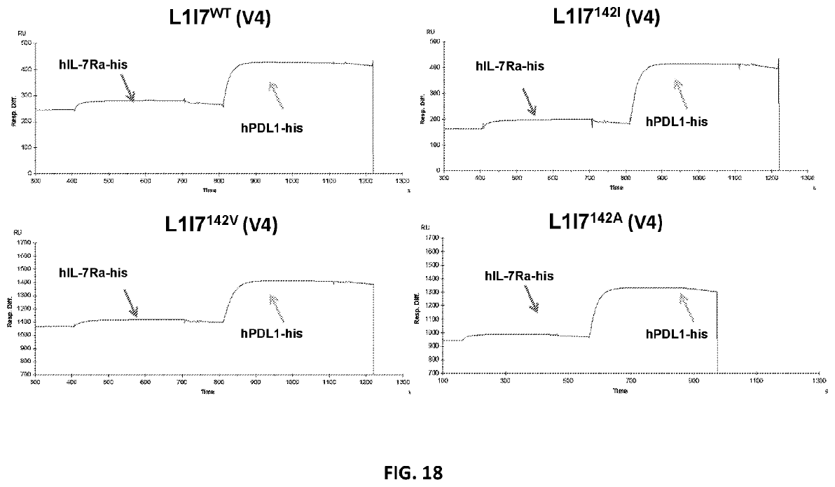

To demonstrate bispecific binding of L1I7 fusion molecules to PD-L1 and IL-7R, this example studied binding kinetics by Biacore T200. Briefly, the L1I7 fusion molecules were captured by Protein A sensor chip. His-tagged IL-7Rα (CD127) and PD-L1 at saturated concentration (100 nM) were sequentially injected over captured antibody at a flow rate of 30 μL/min. The antigens were allowed to associate for 300 s and dissociate for 60 s. The biphasic binding curves shown in FIG. 18 represent the sequential binding of L1I7 to IL-7Rα and PD-L1 for all L1I7 molecules.

### Example 10: Pilot PK/PD Study in Cynomolgus Monkeys of L1I7 Molecules

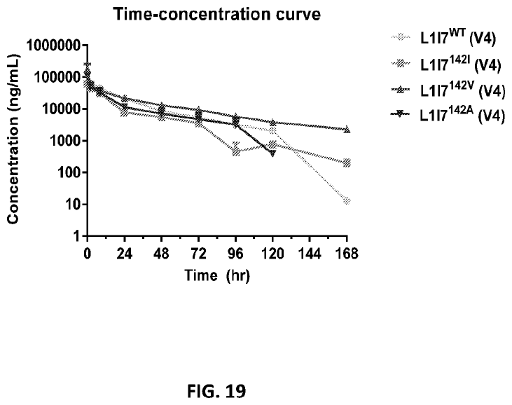

This example studied the PK/PD profile of L1I7 mutant molecules again in a pilot PK setting. Naïve cynomolgus monkeys (n=2, 1 male/1 female) received i.v. injections of L1I7 Format 2 series of molecules (L1I7^{WT}, L1I7^{142I}, L1I7^{142V} and L1I7^{142A}) at 18 mg/kg every week for three times. The dose was chosen as it was close to the clinical efficacious dose for marketed anti-PD-L1 antibodies. Previous PK results of L1I7^{WT} in FIG. 7 showed that L1I7^{WT} became undetectable in the serum by day 7. Thus, the PK parameters obtained from the first dose may be roughly considered as single dose PK parameters in this study. FIG. 19 depicts the time-concentration profiles for L1I7 series of molecules after the first dose. Table 6 lists the PK parameters during the first dosing period. Results from the first dosing period indicated that the systemic clearance of L1I7 molecules was more rapid than a typical IgG. This is consistent with observations with other bifunctional molecules. In addition, t½ for L1I7^{142V} and L1I7^{142A} was relatively longer than that for L1I7^{WT} and L1I7^{142I}, which was consistent with reduced IL-7R internalization by L1I7^{142V} and L1I7^{142A} molecules.

TABLE 6
PK testing results

| Parameters (Units) | L1I7^{WT} | L1I7^{142I} | L1I7^{142V} | L1I7^{142A} |
|---|---|---|---|---|
| T_{1/2} (0-t) (hr) | 17.4 | 16.2 | 42.7 | 24.7 |
| Cₘₐₓ (μg/mL) | 133.1 | 621.9 | 182.0 | 107.2 |
| AUC₍₀₋ₜ₎ (μg*h/mL) | 1671.0 | 922.8 | 1733.6 | 1193.7 |
| CL (mL/hr/kg) | 10.5 | 19.5 | 23.5 | 16.3 |
| Vz/kg (mL/kg) | 268 | 445 | 573 | 627 |

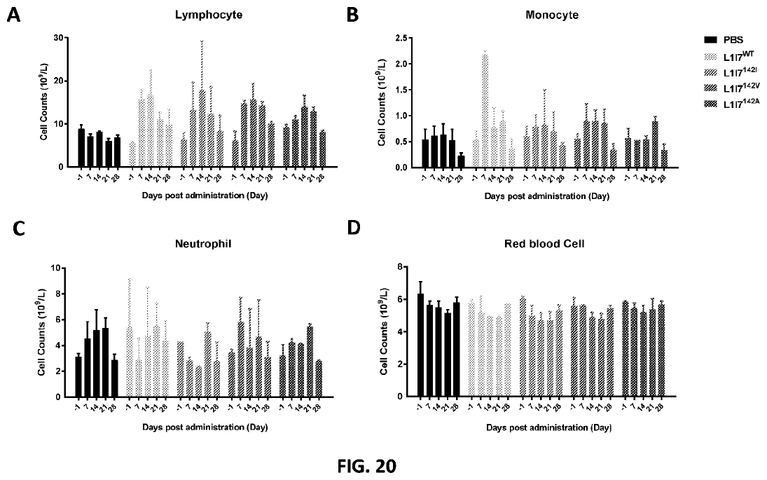

Because IL-7 promotes the survival and proliferation of T lymphocytes, this example took advantage of blood samples from the pilot PK study and monitored absolute lymphocyte count as a quick readout to gauge the pharmacodynamic effect of L1I7 series of molecules with attenuated IL-7 activities. Absolute counts of lymphocytes, neutrophils, monocytes and erythrocytes were examined at pre-dose (Day −1), 7 days after each dose (Day 7, Day 14 and Day 21) and 14 days after the third dose (Day 28) by hematology analyzer ADVIA® 2120 (Siemens). As shown in FIG. 20A, lymphocyte counts increased by up to 2.5 folds of the pre-dose levels at day 7 in L1I7^{WT}- and L1I7^{142I}-treated animals, while L1I7^{142V} and L1I7^{142A} produced less of an effect, with L1I7^{142A} being the weakest. Of note, lymphocyte counts peaked at day 14 (7 days after the second dose). On the other hand, no change was observed in the number of circulating of monocytes, neutrophils or red blood cells in L1I7 molecules except for the wild type which showed a transient surge in monocytes at day 7 (FIG. 20B-D).

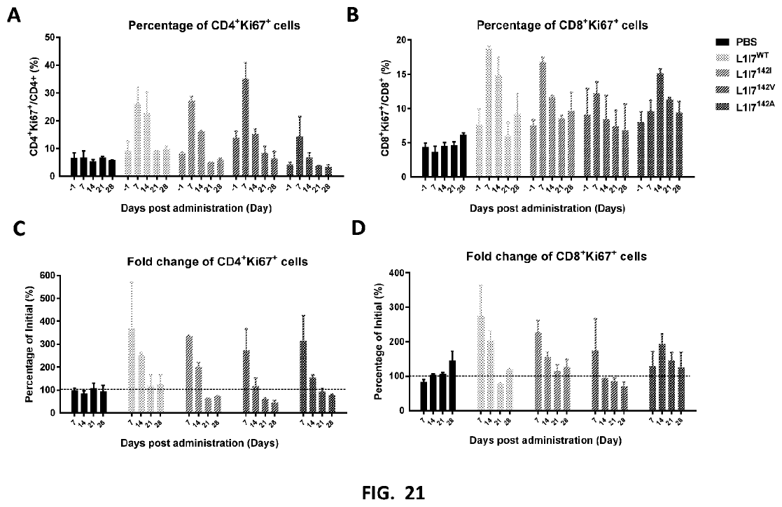

T cell proliferation could be measured by Ki67 expression on CD4⁺ and CD8⁺ T cells. Indeed, Ki67 expression was upregulated relative to pre-dose levels for day 7 and, to a lesser extent, day 14 in both CD4⁺ and CD8⁺ T cells (FIG. 21), and this effect was more pronounced in L1I7^{WT}-treated animals and progressively less so for L1I7^{W142I}-, L1I7^{W142V}- and L1I7^{W142A}-treated animals. These data were consistent with the hematology panel.

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference

1. A polypeptide comprising:
a first portion comprising, in an N-terminal to C-terminal order, a first fragment, a CH2 fragment, and a CH3 fragment, wherein the first fragment comprises an IL-7 protein, an IL-7 protein homologue having at least 75% sequence identity to the IL-7 protein, or a fragment thereof, and wherein the first fragment is capable of binding an IL-7 receptor; and
a second portion comprising, in an N-terminal to C-terminal order, an antigen-binding fragment, a CH2 fragment, and a CH3 fragment, wherein the antigen-binding fragment is capable of specifically binding to a tumor antigen or an immune checkpoint molecule,
wherein the first portion is paired to the second portion through interaction between the CH2 fragments and/or between the CH3 fragments.

2. The polypeptide of claim 1, wherein the first fragment of the first portion has increased or reduced binding affinity to IL-7 receptor alpha, increased or reduced stability, increased or reduced IL-7 activity, increased or reduced IL-7 signaling, or reduced immunogenicity, as compared to the wild-type IL-7 protein.

3. The polypeptide of claim 1, wherein the first fragment of the first portion has reduced binding affinity to IL-7 receptor alpha or reduced IL-7 signaling, as compared to the wild-type IL-7 protein.

4. The polypeptide of claim 1, wherein the first fragment of the first portion comprises the amino acid sequence of SEQ ID NO: 7, or a peptide having at least 75% sequence identity to SEQ ID NO: 7.

5. The polypeptide of claim 4, wherein the peptide has a hydrophobic amino acid residue at position 142 according to numbering of SEQ ID NO:7.

6. The polypeptide of claim 5, wherein the hydrophobic amino acid residue is selected from the group consisting of G, A, V, C, L, I, M, and F.

7. The polypeptide of claim 5, wherein the hydrophobic amino acid residue is selected from the group consisting of A, V, L, I, M, and F.

8. The polypeptide of claim 1, wherein the first fragment of the first portion comprises the amino acid sequence SEQ ID NO:7, 8, 9 or 10.

9. The polypeptide of claim 1, wherein the first fragment of the first portion comprises at least the four alpha-helix motifs of the IL7 protein or the IL-7 homologue.

10. The polypeptide of claim 1, wherein the antigen-binding fragment comprises a Fab fragment, a single-chain variable fragment (scFv), a nanobody, an antigen-binding motif, or a combination thereof.

11. The polypeptide of claim 1, wherein the antigen-binding fragment comprises at least two antigen-binding units.

12. The polypeptide of claim 1, wherein the tumor antigen or immune checkpoint is selected from the group consisting of EGFR, Her2, EpCAM, CD20, CD30, CD33, CD47, CD52, CD133, CD73, CEA, gpA33, Mucins, TAG-72, CIX, PSMA, folate-binding protein, GD2, GD3, GM2, VEGF, VEGFR, Integrin, αVβ3, α5β1, ERBB2, ERBB3, MET, IGF1R, EPHA3, TRAILR1, TRAILR2, RANKL, FAP, Tenascin, PD-L1, PD-1, CTLA-4, LAG-3, CD28, CD122, 4-1BB, TIM3, OX-40, OX40L, CD40, CD40L, LIGHT, ICOS, ICOSL, GITR, GITRL, TIGIT, CD27, VISTA, B7H3, B7H4, HEVM and BTLA.

13. The polypeptide of claim 1, wherein the antigen-binding fragment is capable of specifically binding to a human PD-L1 protein.

14. The polypeptide of claim 13, wherein the antigen-binding fragment comprises a heavy chain variable region (VH) comprising a CDR1, a CDR2, and a CDR3 having the amino acid sequences of residues 31-35, residues 50-66, and residues 99-108 of SEQ ID NO:1, 3, 4 or 5, respectively.

15. The polypeptide of claim 13, wherein the antigen-binding fragment comprises a light chain variable region (VL) comprising a CDR1, a CDR2, and a CDR3 having the amino acid sequences of residues 24-34, residues 50-56, and residues 89-97 of SEQ ID NO:2 or 6, respectively.

16. The polypeptide of claim 15, wherein the VH comprises the amino acid sequence of SEQ ID NO:1, 3, 4 or 5, and the VL comprises the amino acid sequence of SEQ ID NO:2 or 6.

17. The polypeptide of claim 1, wherein the first portion comprises the amino acid sequence of SEQ ID NO:28, 34, 35 or 36.

18. The polypeptide of claim 1, wherein the second portion comprises a chain comprising the amino acid sequence of SEQ ID NO:27, 37 or 31.

19. The polypeptide of claim 18, wherein the second portion further comprises two additional chains each comprising the amino acid sequence of SEQ ID NO:25 or 33.

20. The polypeptide of claim 1,
wherein the first portion comprises the amino acid sequence of SEQ ID NO: 34, the second portion comprises a chain comprising the amino acid sequence of SEQ ID NO: 31, and the second portion further comprises two additional chains each comprising the amino acid sequence of SEQ ID NO: 33;
wherein the first portion comprises the amino acid sequence of SEQ ID NO: 35, the second portion comprises a chain comprising the amino acid sequence of SEQ ID NO: 31, and the second portion further comprises two additional chains each comprising the amino acid sequence of SEQ ID NO: 33;
wherein the first portion comprises the amino acid sequence of SEQ ID NO: 36, the second portion comprises a chain comprising the amino acid sequence of SEQ ID NO: 31, and the second portion further comprises two additional chains each comprising the amino acid sequence of SEQ ID NO: 33;
wherein the first portion comprises the amino acid sequence of SEQ ID NO:34, the second portion comprises a chain comprising the amino acid sequence of SEQ ID NO: 27, and the second portion further comprises two additional chains each comprising the amino acid sequence of SEQ ID NO:25;
wherein the first portion comprises the amino acid sequence of SEQ ID NO: 35, the second portion comprises a chain comprising the amino acid sequence of SEQ ID NO: 27, and the second portion further comprises two additional chains each comprising the amino acid sequence of SEQ ID NO: 25;
wherein the first portion comprises the amino acid sequence of SEQ ID NO: 36, the second portion comprises a chain comprising the amino acid sequence of SEQ ID NO: 27, and the second portion further comprises two additional chains each comprising the amino acid sequence of SEQ ID NO: 25;
wherein the first portion comprises the amino acid sequence of SEQ ID NO:34, the second portion comprises a chain comprising the amino acid sequence of SEQ ID NO: 37, and the second portion further comprises two additional chains each comprising the amino acid sequence of SEQ ID NO:25;
wherein the first portion comprises the amino acid sequence of SEQ ID NO: 35, the second portion comprises a chain comprising the amino acid sequence of SEQ ID NO: 37, and the second portion further comprises two additional chains each comprising the amino acid sequence of SEQ ID NO: 25; or
wherein the first portion comprises the amino acid sequence of SEQ ID NO: 36, the second portion comprises a chain comprising the amino acid sequence of SEQ ID NO: 37, and the second portion further comprises two additional chains each comprising the amino acid sequence of SEQ ID NO: 25.

21. A composition comprising the polypeptide of claim 1 and a pharmaceutically acceptable carrier.

22. An isolated cell comprising one or more polynucleotide encoding the polypeptide of claim 1.

23. A method of treating a cancer in a patient in need thereof, comprising administering to the patient a polypeptide of claim 1.

24. (canceled)

25. The method of claim 23, wherein the cancer is selected from the group consisting of bladder cancer, liver cancer, colon cancer, rectal cancer, endometrial cancer, leukemia, lymphoma, pancreatic cancer, small cell lung cancer, non-small cell lung cancer, breast cancer, urethral cancer, head and neck cancer, gastrointestinal cancer, stomach cancer, oesophageal cancer, ovarian cancer, renal cancer, melanoma, prostate cancer and thyroid cancer.


SEQUENCE LISTING (37 sequences)

SEQ ID NO: 1 - protein, 119 aa, Artificial Sequence
  Synthetic
EVQLVESGGGLVQPGGSLRLSCAASGFTFSSYDMSWVRQAPGKSLEWVATISDAGGYIYYRDSVKGRFTISRDNAKNSLYLQMNSLRDEDTAVYICARELPWRYALDYWGQGTTVTVSS

SEQ ID NO: 2 - protein, 107 aa, Artificial Sequence
  Synthetic
DIQMTQSPSSLSASVGDRVTITCKASQDVTPAVAWYQQKPGKAPKLLIYSTSSRYTGVPSRFSGSGSGTDFTFTISSLQPEDIATYYCQQHYTTPLTFGQGTKLEIK

SEQ ID NO: 3 - protein, 119 aa, Artificial Sequence
  Synthetic
EVQLVESGGGLVQPGGSLRLSCAASGFTFSSYDMSWVRQAPGKSLEWVATISDAGGYIYYRDSVKGRFTISRDNAKNSLYLQMNSLRDEDTAVYICAREFGKRYALDYWGQGTTVTVSS

SEQ ID NO: 4 - protein, 119 aa, Artificial Sequence
  Synthetic
EVQLVESGGGLVQPGGSLRLSCAASGFTFSSYDMSWVRQAPGKSLEWVATISDAGGYIYYSDSVKGRFTISRDNAKNSLYLQMNSLRDEDTAVYICAREFGKRYALDYWGQGTTVTVSS

SEQ ID NO: 5 - protein, 119 aa, Artificial Sequence
  Synthetic
QVQLLESGGGLVQPGGSLRLSCAASGFTFSSYWMSWVRQAPGKGLEWVANIKQDGSEKYYVDSVKGRFTISRDNAKNSLYLQMNSLRAEDTAVYYCARVALWDDAFDIWGQGTMVTVSS

SEQ ID NO: 6 - protein, 107 aa, Artificial Sequence
  Synthetic
DIQMTQSPSTLSASVGDRVIITCRASRGISSWLAWYQQKPGKAPNLLISKASSLESGVPSRFSGSGSGTDFTLTISSLQPEDFATYYCQQSSSIPLTFGGGTKVEIK

SEQ ID NO: 7 - protein, 152 aa, Artificial Sequence
  Synthetic
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 8 - protein, 152 aa, Artificial Sequence
  Synthetic
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCINKILMGTKEH

SEQ ID NO: 9 - protein, 152 aa, Artificial Sequence
  Synthetic
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCVNKILMGTKEH

SEQ ID NO: 10 - protein, 152 aa, Artificial Sequence
  Synthetic
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCANKILMGTKEH

SEQ ID NO: 11 - protein, 30 aa, Artificial Sequence
  Synthetic
GGGGSGGGGSGGGGSGGGGSGGGGSGGGGS

SEQ ID NO: 12 - protein, 107 aa, Artificial Sequence [sequence omitted]

SEQ ID NO: 13 - protein, 98 aa, Artificial Sequence
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 14 - protein, 217 aa, Artificial Sequence
  Synthetic
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 217 aa, Artificial Sequence
  Synthetic
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 217 aa, Artificial Sequence
  Synthetic
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 17 - protein, 5 aa, Artificial Sequence
  Synthetic
EPKSC

SEQ ID NO: 18 - protein, 15 aa, Artificial Sequence
  Synthetic
EPKSCDKTHTCPPCP

SEQ ID NO: 19 - protein, 15 aa, Artificial Sequence
  Synthetic
EPKSADKTHTCPPCP

SEQ ID NO: 20 - protein, 5 aa, Artificial Sequence
  Synthetic
EPKSA

SEQ ID NO: 21 - protein, 631 aa, Artificial Sequence
  Synthetic
EVQLVESGGGLVQPGGSLRLSCAASGFTFSSYDMSWVRQAPGKSLEWVATISDAGGYIYYRDSVKGRFTISRDNAKNSLYLQMNSLRDEDTAVYICARELPWRYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 22 - protein, 631 aa, Artificial Sequence
  Synthetic
EVQLVESGGGLVQPGGSLRLSCAASGFTFSSYDMSWVRQAPGKSLEWVATISDAGGYIYYRDSVKGRFTISRDNAKNSLYLQMNSLRDEDTAVYICARELPWRYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCINKILMGTKEH

SEQ ID NO: 23 - protein, 631 aa, Artificial Sequence
  Synthetic
EVQLVESGGGLVQPGGSLRLSCAASGFTFSSYDMSWVRQAPGKSLEWVATISDAGGYIYYRDSVKGRFTISRDNAKNSLYLQMNSLRDEDTAVYICARELPWRYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCVNKILMGTKEH

SEQ ID NO: 24 - protein, 631 aa, Artificial Sequence
  Synthetic
EVQLVESGGGLVQPGGSLRLSCAASGFTFSSYDMSWVRQAPGKSLEWVATISDAGGYIYYRDSVKGRFTISRDNAKNSLYLQMNSLRDEDTAVYICARELPWRYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCANKILMGTKEH

SEQ ID NO: 25 - protein, 214 aa, Artificial Sequence
  Synthetic
DIQMTQSPSSLSASVGDRVTITCKASQDVTPAVAWYQQKPGKAPKLLIYSTSSRYTGVPSRFSGSGSGTDFTFTISSLQPEDIATYYCQQHYTTPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 631 aa, Artificial Sequence
  Synthetic
EVQLVESGGGLVQPGGSLRLSCAASGFTFSSYDMSWVRQAPGKSLEWVATISDAGGYIYYRDSVKGRFTISRDNAKNSLYLQMNSLRDEDTAVYICAREFGKRYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 27 - protein, 671 aa, Artificial Sequence
  Synthetic
EVQLVESGGGLVQPGGSLRLSCAASGFTFSSYDMSWVRQAPGKSLEWVATISDAGGYIYYSDSVKGRFTISRDNAKNSLYLQMNSLRDEDTAVYICAREFGKRYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCEVQLVESGGGLVQPGGSLRLSCAASGFTFSSYDMSWVRQAPGKSLEWVATISDAGGYIYYSDSVKGRFTISRDNAKNSLYLQMNSLRDEDTAVYICAREFGKRYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 384 aa, Artificial Sequence
  Synthetic
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHEPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 29 - protein, 631 aa, Artificial Sequence
  Synthetic
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGGGSGGGGSGGGGSGGGGSGGGGSGGGGSEVQLVESGGGLVQPGGSLRLSCAASGFTFSSYDMSWVRQAPGKSLEWVATISDAGGYIYYSDSVKGRFTISRDNAKNSLYLQMNSLRDEDTAVYICAREFGKRYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 631 aa, Artificial Sequence
  Synthetic
QVQLLESGGGLVQPGGSLRLSCAASGFTFSSYWMSWVRQAPGKGLEWVANIKQDGSEKYYVDSVKGRFTISRDNAKNSLYLQMNSLRAEDTAVYYCARVALWDDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 31 - protein, 671 aa, Artificial Sequence
  Synthetic
QVQLLESGGGLVQPGGSLRLSCAASGFTFSSYWMSWVRQAPGKGLEWVANIKQDGSEKYYVDSVKGRFTISRDNAKNSLYLQMNSLRAEDTAVYYCARVALWDDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCQVQLLESGGGLVQPGGSLRLSCAASGFTFSSYWMSWVRQAPGKGLEWVANIKQDGSEKYYVDSVKGRFTISRDNAKNSLYLQMNSLRAEDTAVYYCARVALWDDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 631 aa, Artificial Sequence
  Synthetic
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGGGSGGGGSGGGGSGGGGSGGGGSGGGGSQVQLLESGGGLVQPGGSLRLSCAASGFTFSSYWMSWVRQAPGKGLEWVANIKQDGSEKYYVDSVKGRFTISRDNAKNSLYLQMNSLRAEDTAVYYCARVALWDDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 214 aa, Artificial Sequence
  Synthetic
DIQMTQSPSTLSASVGDRVIITCRASRGISSWLAWYQQKPGKAPNLLISKASSLESGVPSRFSGSGSGTDFTLTISSLQPEDFATYYCQQSSSIPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 384 aa, Artificial Sequence
  Synthetic
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCINKILMGTKEHEPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 35 - protein, 384 aa, Artificial Sequence
  Synthetic
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCVNKILMGTKEHEPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 36 - protein, 384 aa, Artificial Sequence
  Synthetic
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCANKILMGTKEHEPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 37 - protein, 671 aa, Artificial Sequence
  Synthetic
EVQLVESGGGLVQPGGSLRLSCAASGFTFSSYDMSWVRQAPGKSLEWVATISDAGGYIYYRDSVKGRFTISRDNAKNSLYLQMNSLRDEDTAVYICARELPWRYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCEVQLVESGGGLVQPGGSLRLSCAASGFTFSSYDMSWVRQAPGKSLEWVATISDAGGYIYYSDSVKGRFTISRDNAKNSLYLQMNSLRDEDTAVYICAREFGKRYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK